(19) United States
(12) Patent Application Publication
(10) Pub. No.: US 2026/0053751 A1
(43) Pub. Date: Feb. 26, 2026

(54) Dry Powdered Formulations

(71) Applicant: Pinata Holdings Inc., Los Angeles, CA (US)

(72) Inventors: Michael OGBURN, Lake Forest, CA (US); Christopher PRICE, Monroe Township, NJ (US)

(21) Appl. No.: 19/252,506

(22) Filed: Jun. 27, 2025

Related U.S. Application Data
(63) Continuation of application No. 18/822,564, filed on Sep. 3, 2024, now abandoned.
Application No. 18/822,564 is a continuation of application No. PCT/US2023/063548, filed on Mar. 2, 2023.
(60) Provisional application No. 63/316,629, filed on Mar. 4, 2022.
(60) Provisional application No. 63/318,827, filed on Mar. 11, 2022.
(60) Provisional application No. 63/425,400, filed on Nov. 15, 2022.

(51) Int. Cl. A61K 9/50; A61K 31/00

(52) U.S. Cl. CPC A61K 9/5042; A61K 9/5026; A61K 9/5073; A61K 31/658

(57) ABSTRACT

Provided herein are pharmaceutical compositions, kits comprising pharmaceutical compositions, methods of treating disease, and methods of making compositions and kits described herein. The pharmaceutical compositions described herein are powdery pharmaceutical compositions. The powdery pharmaceutical compositions herein can contain particles that are encapsulated with multiple layers of material.

### CROSS-REFERENCE

This application is a continuation of U.S. application Ser. No. 18/822,564, filed Sep. 3, 2024, which is a continuation of National Stage Entry of International Application No. PCT/US2023/063548, filed Mar. 2, 2023, which claims the benefit of U.S. Provisional Application No. 63/316,629, filed Mar. 4, 2022, of U.S. Provisional Application No. 63/318,827, filed Mar. 11, 2022, and of U.S. Provisional Application No. 63/425,400, filed Nov. 15, 2022, the disclosures of which are incorporated herein by reference in their entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are powdery compositions, comprising: a plurality of encapsulated spray dried particles. In some embodiments, each particle of the plurality of spray dried particles can be substantially encapsulated by a first coating. In some embodiments, the first coating can be substantially encapsulated by one or more additional coatings. In some embodiments, the plurality of encapsulated spray dried particles can comprise a cannabinoid or a pharmaceutically acceptable salt thereof. In some embodiments, within the plurality of encapsulated spray dried particles, the plurality of encapsulated spray dried particles individually have a particle diameter ranging from about 20 micrometers to about 200 micrometers, or have a mean or median particle diameter of about 20 micrometers to about 200 micrometers, as measured by a particle analyzer using laser diffraction. In some embodiments, the first coating can comprise a hydroxypropyl methylcellulose (HPMC), a hydroxypropyl methylcellulose acetate succinate (HPMCAS), a cyclodextrin, a maltodextrin, a povidone, a copovidone or any combination thereof. In some embodiments, the one or more additional coatings can comprise an enteric coating. In some embodiments, an enteric coating can comprise a polymer. In some embodiments, an enteric coating can comprise a methyl methacrylate (MMA). In some embodiments, a cannabinoid or a pharmaceutical acceptable salt thereof can be present in an amount ranging from about 1 mg to about 100 mg. In some embodiments, the cannabinoid or the pharmaceutical acceptable salt thereof can be in the form of an oil. In some embodiments, the cannabinoid or the pharmaceutically acceptable salt thereof can comprise a tetrahydrocannabinol (THC) or a pharmaceutically acceptable salt thereof. In some embodiments, the tetrahydrocannabinol (THC) or the pharmaceutical acceptable salt thereof can comprise tetrahydrocannabinol Delta-8, tetrahydrocannabinol Delta-9, tetrahydrocannabinol Delta-10, tetrahydrocannabinol Delta-11, tetrahydrocannabinol Delta-13, tetrahydrocannabivarin (THCV), tetrahydrocannabinolic acid (THCA), full spectrum THC, broad spectrum THC, or a pharmaceutically acceptable salt thereof. In some embodiments, the cannabinoid or the pharmaceutically acceptable salt thereof can comprise a cannabidiol (CBD), a full spectrum CBD, a broad spectrum CBD, or a pharmaceutically acceptable salt thereof. In some embodiments, the cannabinoid or the pharmaceutically acceptable salt thereof can comprise cannabigerol (CBG), cannabichromene (CBC), cannabidivarin (CBDV), cannabinol (CBN) a pharmaceutically acceptable salt of any of these, or any combination thereof. In some embodiments, the composition can further comprise a second cannabinoid or a pharmaceutically acceptable salt thereof. Also disclosed herein are kits comprising the powdery composition. Also disclosed herein are liquids comprising the powdery composition. In some embodiments, the liquid can be a beverage. Also disclosed herein are dietary supplements comprising the powdery composition. Also disclosed herein are foods comprising the powdery composition. In some embodiments, the food can be a medical food. Also disclosed herein are methods of making the composition. In some embodiments, a method can comprise: contacting the powdery composition with a liquid, an excipient, a carrier, a diluent, a food, a medical food, a capsule, a vitamin, an unsaturated fatty acid, an ester thereof, or a salt thereof, an organic salt, an inorganic salt, or any combination thereof.

Also disclosed herein are pharmaceutical compositions. In some embodiments, a composition disclosed herein can be a powdery pharmaceutical composition. In some embodiments, a pharmaceutical composition herein can further comprise particles of an pharmaceutically acceptable excipient. In some embodiments, a powdery pharmaceutical composition cam be in unit dose form. In some embodiments, a plurality of encapsulated spray dried particles can be contained within a container. In some embodiments, a container can be about one quarter to about one half, by volume, filled with the powdery pharmaceutical composition. In some embodiments, a portion of the container not containing the powdery pharmaceutical composition can comprise a gas that at least partially comprises an inert gas. In some embodiments, the inert gas can comprise nitrogen, carbon dioxide, helium, or any combination thereof. In some embodiments, the inert gas can comprise at least about: 80%, 85%, 90%, or 95% of the gas on a volume to volume basis. In some embodiments, at least a portion of the particles of the pharmaceutically acceptable excipient individually have a particle diameter ranging from about 50 micrometers to about 200 micrometers, or have a mean or median particle diameter of about 50 micrometers to about 200 micrometers, as measured by a particle analyzer using laser diffraction. In some embodiments, the plurality of encapsulated spray dried particles and the particles of the pharmaceutically acceptable excipient can be admixed into a substantially homologous mixture. In some embodiments, a weight to weight ratio of the plurality of encapsulated spray dried particles and the particles of the pharmaceutically acceptable excipient, can range from about 1:1 (w/w) to about 10000:1 (w/w). In some embodiments, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. Also disclosed herein are kits comprising a powdery pharmaceutical composition.

Also disclosed herein are methods of treating a disease or condition in a subject in need thereof. In some embodiments, a method can comprise treating the disease or condition by orally administering a therapeutically effective amount of a powdery pharmaceutical composition disclosed herein. In some embodiments, administering can be conducted one, twice, three, or four times per day. In some embodiments, the disease or condition can be selected from the group consisting of a cancer, a breast cancer, a melanoma, an anxiety, a pruritus (itching), a cognitive function, Alzheimer's disease, amyotrophic lateral sclerosis, Huntington's disease, Parkinson's disease, a chronic pain, pain management, multiple sclerosis, a side effect of chemotherapy, HIV, AIDS, a neurodegenerative disorder, a behavior disorder, Tourette syndrome, cervical dystonia, sleep disorder, an appetite disorder, a seizure, an epilepsy, nausea associated with chemotherapy, an anorexia, a spinal cord injury, a glaucoma, a schizophrenia, an epilepsy, an asthma, a posttraumatic stress disorder, cachexia, irritable bowel syndrome, a substance dependency disorder, a psychiatric symptom, an autoimmune disease, an inflammation, a sleep apnea, a headache, a migraine, an opioid addiction, and any combination thereof. In some embodiments, a powdery pharmaceutical composition can be administered as needed, or for about: one day, two days, three days, four days, five days, six days, a week, two weeks, three weeks, a month, two months, three months, four months, five months, six months, seven months, eight months, nine months, ten months, eleven months, a year, or chronically. In some embodiments, an amount of a cannabinoid or a pharmaceutically acceptable salt thereof can range from about 1 mg to about 25 mg. In some embodiments, a second therapeutic or a pharmaceutically acceptable salt thereof can be administered. In some embodiments, a second therapeutic or a pharmaceutically acceptable salt thereof can be administered concurrently or consecutively. In some embodiments, a subject can be diagnosed with a disease or condition. In some embodiments, a powdery pharmaceutical composition can be administered in the form of a beverage or a food.

Also disclosed herein are methods of making coated microencapsulated particles. In some embodiments, the method can comprise applying a second coating in a fluid bed wherein: i) the microencapsulated particles comprise a spray dried and encapsulated cannabinoid or a salt thereof; ii) wherein the second coating comprises an enteric coating.

### BRIEF DESCRIPTION OF THE DRAWINGS

FIG. 1 shows a spray drying manufacturing system comprising a closed spray drying chamber which receives a solution comprising an active ingredient microencapsulated in one or more layers of a polymer in a suitable solvent. The system generates dried microencapsulated particles with one or more layers from the solution comprising the microencapsulated active ingredient to enhance bioavailability of the low soluble active ingredient.

### DETAILED DESCRIPTION

### Overview

Delivering compositions through oral ingestion can result in uneven bioavailability of the active ingredient. The absorption through stomach may take longer if fatty foods are eaten prior to ingestion of the active ingredient, further slowing down the process. By spray drying the compositions and applying an enteric coating in a fluid bed or via another process, the particles can bypass the stomach acids and release within the gastrointestinal tract, resulting in more consistent and higher bioavailability. The particles can contain multiple layers of an encapsulation material, for example the particles can comprise one or more coatings. The encapsulated particles can be added to a food or beverage. The time needed for the pharmaceutical to reach the blood stream can be reduced depending on food intake. In addition, the dosing level can also be reduced as compared to a gelatin gummy, tincture, oral tablet or capsule equivalent

Provided herein are compositions, unit dose kits comprising compositions, methods of treating and preventing disease, and methods of making compositions and kits described herein. Pharmaceutical drugs described herein can be produced employing various methods to synthesize, manipulate, and administer particles. In some embodiments, the compositions described herein are powdery compositions. In some embodiments, the powdery compositions described herein are powdery pharmaceutical compositions. In some embodiments, the powdery compositions described herein are dietary supplement compositions.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject may not be necessarily diagnosed or suspected of being at high risk for the disease.

The term “substantially” or “essentially” can refer to a qualitative condition that exhibits an entire or nearly total range or degree of a feature or characteristic of interest. In some cases, substantially encapsulated can refer to near complete encapsulation of a substance or compound. For example, substantially encapsulated can comprise a particle that is at least about: 70%, 75%, 80%, 85%, 90%, 95%, 99%, or 100% encapsulated. In some cases, substantially can refer to at least about: 70%, 75%, 80%, 85%, 90%, 95%, 99%, or 100% of the total range or degree of a feature or characteristic of interest. In some cases, the term “substantially” can refer to a degree of deviation that is sufficiently small so as to not measurably detract from the identified property or circumstance. In some cases, the exact degree of deviation allowable may in some cases depend on the specific context.

The term “at least partially” can refer to a qualitative condition that exhibits a partial range or degree of a feature or characteristic of interest. In some cases, at least partially encapsulated can refer to a partial encapsulation of a substance or compound. For example, at least partially encapsulated can comprise a particle that is at least about: 5%, 10%, 20%, 30%, 40%, 50%, 60%, 70%, 75%, 80%, 85%, 90%, 95%, 99%, or 100% encapsulated.

The term “in vivo” can be used to describe an event that takes place in a subject's body.

The term “ex vivo” can be used to describe an event that takes place outside of a subject's body. An “ex vivo” assay may not be performed on a subject. Rather, it can be performed upon a sample separate from a subject. An example of an “ex vivo” assay performed on a sample can be an “in vitro” assay.

The term “in vitro” can be used to describe an event that takes place contained in a container for holding laboratory reagent such that it can be separated from the living biological source organism from which the material may be obtained. In vitro assays can encompass cell-based assays in which cells alive or dead are employed. In vitro assays can also encompass a cell-free assay in which no intact cells are employed.

As used herein, the term “about” a number can refer to that number plus or minus 10% of that number. The term “about” a range can refer to that range minus 10% of its lowest value and plus 10% of its greatest value. In some cases, the term “about” a number can refer to that number plus or minus 20% of that number. The term “about” a range can refer to that range minus 20% of its lowest value and plus 20% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “unit dose” or “dosage form” can be used interchangeably and can be meant to refer to pharmaceutical drug products in the form in which they are marketed for use, with a specific mixture of active ingredients and inactive components or excipients, in a particular configuration, and apportioned into a particular dose to be delivered. The term “unit dose” can also sometimes encompass non-reusable packaging, although the FDA distinguishes between unit dose “packaging” or “dispensing”. More than one unit dose can refer to distinct pharmaceutical drug products packaged together, or to a single pharmaceutical drug product containing multiple drugs and/or doses. The term “unit dose” can also sometimes refer to the particles comprising a pharmaceutical composition, and to any mixtures involved. Types of unit doses may vary with the route of administration for drug delivery, and the substance(s) being delivered. A solid unit dose can be the solid form of a dose of a chemical compound used as a pharmaceutically acceptable drug or medication intended for administration or consumption.

As used herein, the term “fine particle fraction” or “fine particle fraction from the emitted dose” can refer to the mass of active agent having an aerodynamic diameter below about: 5 μm, 6 μm, 7 μm, 8 μm, 9 μm, or 10 μm. In some instances, the cutoff size can be less than or equal to an aerodynamic diameter of about 5 μm. In some instances, the cutoff size can be less than or equal to an aerodynamic diameter of about 6.4 μm. In some instances, the cutoff size can be less than or equal to an aerodynamic diameter of about 7 μm or about 8 μm. In some instances, the fine particle fraction can be often used to evaluate the efficiency of aerosol deaggregation. In some cases, fine particle fraction can be the mass of active agent having an aerodynamic diameter below about: 5 μm, 6 μm, 7 μm, 8 μm, 9 μm, or 10 μm as a percentage of an emitted dose mass. For example, a composition described herein can have a fine particle fraction of at least about: 20%, 30%, 40%, 50%, 60%, 70%, 80%, or 90% upon aerosolization.

As used herein, a “dose” can refer to a measured quantity of a therapeutic agent to be taken at one time.

As used herein, “pharmaceutically acceptable salt” can refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, “laser diffraction” can refer to a method for particle size analysis, which consists of scattering laser light off an assembly of particles, and collecting the scattered light using a spatial array of detectors. The signal from the detectors can be a pattern of scattered/diffracted light vs. angle. This pattern can result from many particles being illuminated by the laser light source at the same time, where all of their individual scattered/diffracted light rays mix together at each detector element.

As used herein, “particle size analyzer” can refer to an instrument for particle size analysis, particle size measurement, or simply particle sizing.

As used herein, “particle size analysis” can refer to the collective name of the technical procedures, or laboratory techniques which determines the size range, and/or the average (mean), median or mode size of the particles in a powder or liquid sample.

As used herein, “time to peak plasma concentration” can refer to the time required for a drug to reach peak concentration in plasma. Peak concentration in plasma can be usually defined as the plasma concentration that a drug achieves in a specified compartment or test area of the body after the drug has been administered and before the administration of a second dose.

As used herein, “HPLC” can refer to high-performance liquid chromatography (formerly referred to as high-pressure liquid chromatography), which is a technique in analytical chemistry used to separate, identify, and quantify each component in a mixture. HPLC can be a common technique used in pharmaceutical development, as it can be a method to ensure product purity.

As used herein, the terms “effective amount” or “therapeutically effective amount” of a drug used to treat a disease can be an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose.

As used herein, the terms “fluid bed” or “fluidized bed” can refer to a process in which an active ingredient that has been spray dried and encapsulated to form particles or granules, is processed through a piece of equipment (e.g., a fluidized bed reactor). The process comprises spraying a polymer (barrier, enteric coating) binder as a solution, suspension, or melt onto a fluidized powder bed. The process can produce one or more additional coatings on a microencapsulated particle.

As used herein, the term “milling” can be the process in which a piece of equipment physically breaks down coarse particles to finer particles to achieve a tight particle size distribution.

The section headings used herein are for organizational purposes only and are not to be construed as limiting the subject matter described.

### Compositions

Disclosed herein are devices, systems and methods for producing, packaging, and delivering stable powdery compositions via oral administration. In some embodiments, the compositions can be spray dried. In some embodiments, the spray dried powder can be processed through a fluid bed to apply a polymer barrier or enteric coating. In some embodiments, a composition can comprise a pharmaceutical composition or a dietary supplement composition. In some embodiments, a pharmaceutical composition can comprise a composition. In some embodiments, the compositions can comprise one or more coatings. In those embodiments, the addition of an excipient carrier product to the active pharmaceutical powders can be used to improve its stability and effective solubility.

In some embodiments, a composition herein can comprise a plurality of encapsulated spray dried particles, wherein each particle of the plurality of spray dried particles can substantially encapsulated by a first coating. In some cases, the first coating can be substantially encapsulated by one or more additional coatings. In some cases, the plurality of encapsulated spray dried particles can comprise a cannabinoid or a pharmaceutically acceptable salt thereof. In some cases, the plurality of encapsulated spray dried particles can individually have a particle diameter ranging from about 20 micrometers to about 200 micrometers, or have a mean or median particle diameter of about 20 micrometers to about 200 micrometers, as measured by a particle analyzer using laser diffraction. In some cases, the first coating can comprise a hydroxypropyl methylcellulose (HPMC), a hydroxypropyl methylcellulose acetate succinate (HPMCAS), a cyclodextrin, a maltodextrin, a povidone, a copovidone or any combination thereof. In some cases, the one or more additional coatings comprise an enteric coating.

In some embodiments, a liquid can comprise a composition herein. In some cases, a liquid can be a beverage. In some cases, a food can comprise a composition herein. In some cases, a food can be a medical food.

In some embodiments the compositions can comprise one or more of: an active ingredient or salts, excipients, and inactive ingredients. For example, a composition disclosed herein can comprise 1, 2, 3, 4, 5, 6, or more cannabinoids. In some cases, a composition can comprise particles. In some cases, particles can comprise an excipient (e.g., a pharmaceutically acceptable excipient) or an active ingredient. In some cases, the compositions can comprise a pharmaceutical composition. In some instances, a composition can comprise particles of a pharmaceutically acceptable excipient. In some instances, a composition can comprise particles comprising an active ingredient or a pharmaceutically acceptable salt thereof at least partially encapsulated in a coating material and wherein the particles at least partially encapsulated in the coating material are spray dried. In some instances, particles can comprise more than one coating. In some instances the encapsulated particles can be processed through a fluid bed to apply a polymer barrier or enteric coating.

As used herein, “coating material” can refer to a material added via a pharmaceutical coating process by which an essentially dry, outer layer of coating material can be applied to the surface of a dosage form or to a layer of a previously applied coating material. Coating dosage forms may be used to improve stability (light protection, moisture and gas barrier), facilitate administration, or modify the drug release behavior from the dosage form. The coating materials may be used to enable the immediate release of the drug, delay the release of the drug (such as in enteric coatings), or sustain the release of the drug from the dosage form over extended periods of time. Coating materials may include film coating formulations, which usually contain a polymer, a plasticizer, a colorant, opacifier, a solvent, and a vehicle. In some cases, a coating material can refer to the coating material used in the coating of a particle of an active ingredient to create an encapsulated particle.

In some embodiments, a composition can comprise a mixture of particles described herein. In some embodiments, the particles can be mixed in a substantially homogenous mixture, for example. an excipient and a composition described herein can be admixed into a substantially homologous mixture. In some instances, at least a portion of the particles of an excipient can have a particle diameter ranging from about 50 micrometers to about 200 micrometers, as measured by a particle size analyzer using laser diffraction; at least a portion of the particles comprising an active ingredient or a pharmaceutically acceptable salt thereof at least partially encapsulated in a coating material can have a particle diameter ranging from about 20 micrometers to about 200 micrometers, or about 40 micrometers to about 70 micrometers, as measured by a particle size analyzer using laser diffraction; and wherein in a human clinical trial, the powdery composition, when taken orally can provide in at least part of the humans in the clinical trial a time to peak plasma concentration (Tmax) of the active ingredient or the salt thereof ranging from about 10 minutes to about 120 minutes, or from about 15 minute to about 45 minutes. In some embodiments, the Tmax of the active ingredient or the salt thereof ranging from about 5 min to about 10 min, about 10 min to about 15 min, about 10 min to about 20 min, about 10 min to about 25 min, about 10 min to about 30 min, about 10 min to about 40 min, about 10 min to about 50 min, about 10 min to about 60 min, about 5 min to about 10 min, about 5 min to about 20 min, about 5 min to about 25 min, about 5 min to about 30 min, about 5 min to about 40 min, about 5 min to about 50 min, about 5 min to about 60 min, about 10 min to about 20 min, about 10 min to about 25 min, about 10 min to about 30 min, about 10 min to about 40 min, about 10 min to about 50 min, about 10 min to about 60 min, about 20 min to about 25 min, about 20 min to about 30 min, about 20 min to about 40 min, about 20 min to about 50 min, about 20 min to about 60 min, about 25 min to about 30 min, about 25 min to about 40 min, about 25 min to about 50 min, about 25 min to about 60 min, about 30 min to about 40 min, about 30 min to about 50 min, about 30 min to about 60 min, about 40 min to about 50 min, about 40 min to about 60 min, or about 50 min to about 60 min.

In some embodiments, through oral administration in a human clinical trial, the powdery pharmaceutical composition operates mechanistically such that in at least a portion of the humans in the clinical trial, a majority of the particles bypass the stomach and release in the gastrointestinal tract.

In some embodiments, the weight to weight ratio of a) the particles of a pharmaceutically acceptable excipient and b) particles comprising an active ingredient or a pharmaceutically acceptable salt thereof at least partially encapsulated in two or more coating materials ranges from about 1:1 to about 10000:1. In some embodiments, the weight to weight ratio of: a) the particles of a pharmaceutically acceptable excipient and b) particles comprising an active ingredient or a pharmaceutically acceptable salt thereof at least partially encapsulated in two or more coating materials can range from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some embodiments, the weight to weight ratio of: a) the particles of a pharmaceutically acceptable excipient and b) particles comprising an active ingredient or a pharmaceutically acceptable salt thereof at least partially encapsulated in a coating material can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some embodiments, the weight to weight ratio of: a) the particles of a pharmaceutically acceptable excipient and b) particles comprising an active ingredient or a pharmaceutically acceptable salt thereof at least partially encapsulated in two or more coating materials can range from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10. In some embodiments, an active ingredient or a pharmaceutically acceptable salt thereof (e.g. a cannabinoid or the pharmaceutically acceptable salt thereof) can comprise at least about: 1%, 2%, 3%, 4%, 5%, 6%, 7%, 8%, 9%, or 10% by weight of a pharmaceutical composition.

In some embodiments, at least a portion of the particles of a pharmaceutical excipient and particles comprising an active ingredient or a pharmaceutically acceptable salt thereof at least partially encapsulated in two or more coating materials may not be covalently bound to each other.

In some aspects, a solid active ingredient is spherical or substantially spherical and is not encapsulated. In other cases, the solid active ingredient is spherical or substantially spherical and is encapsulated. In some aspects, an active ingredient is spherical or substantially spherical and is not encapsulated. In some cases, the active ingredient is spherical or substantially spherical and is encapsulated. In certain cases, the spherical or substantially spherical active ingredient is mixed with an excipient. In certain cases, the excipient can also be spherical or substantially spherical. In certain cases, the excipient may comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, fumaryl diketopiperazine (FDKP) a pharmaceutically acceptable salt of any of these, or any combination thereof.

### Active Ingredients and Pharmaceutically Acceptable Salts

An active pharmaceutical ingredient can be any substance or mixture of substances intended to be used in the manufacture of a drug (medicinal) product and that, when used in the production of a drug, becomes an active ingredient of the drug product. Such substances can be intended to furnish pharmacological activity or other direct effect in the diagnosis, cure, mitigation, treatment, or prevention of disease or to affect the structure or function of the body. In some cases, an active ingredient can comprise a pharmaceutical compound. In some cases, a pharmaceutical compound can comprise an active ingredient.

In some embodiments, an active pharmaceutical ingredient or salt thereof can be formulated as oil emulsion. In some embodiments, an active pharmaceutical ingredient or salt thereof can be formulated as an oil, a liquid, or a gel. In some embodiments, the active pharmaceutical ingredient or salt thereof can be encapsulated in a coating material and can be spray dried. In some embodiments, the active pharmaceutical ingredient or salt thereof can be encapsulated in two or more coating materials and can be spray dried. In some cases, at least one of the coating materials can be an enteric coating material.

In some embodiments, the active pharmaceutical ingredients or salts thereof can comprise cannabinoids or salts thereof. The term “cannabinoid” can refer to a chemical compound that shows direct or indirect activity at a cannabinoid receptor. In some instances, a cannabinoid can comprise a phytocannabinoid. In some instances, a cannabinoid can comprise a endocannabinoid. In some instances, an endocannabinoid can comprise anandamide (arachidonoyl ethanolamide) or 2-arachidonoyl glycerol (2-AG). In some cases, a cannabinoid can be a full spectrum cannabinoid. In some cases, a cannabinoid can be a broad-spectrum cannabinoid. In some cases, a cannabinoid can be a an isolated cannabinoid. Examples of cannabinoids include, but are not limited to, tetrahydrocannabinol (THC), cannabidiol (CBD), cannabinol (CBN), cannabigerol (CBG), cannabichromene (CBC), cannabicyclol (CBL), cannabivarin (CBV), cannabidivarin (CBDV), cannabichromevarin (CBCV), cannabigerovarin (CBGV), cannabigerol monomethyl ether (CBGM), and cannabidiolic acid (CBDA). In some cases, a cannabinoid can comprise cannabielsoin (CBE), cannabicitran (CBT), 10-Oxo-delta-6a-tetrahydrocannabinol (OTHC), cannabichromanon, cannabifuran, cannabiglendol, cannabiripsol, or cannbicitran. In some cases, a cannabinoid can comprise a cannabinoid from Table 1.

TABLE 1
List of exemplary cannabinoids

| Cannabinoid | Abbreviation |
|---|---|
| Cannabigerovarin | CBGV |
| Cannabigerovarinic acid | CBGV A |
| Cannabigerol | CBG |
| Cannabigerolic acid | CBGA |
| O-Methlycannabigerol | |
| Cannabigerolic acid methylether | |
| Cannabinerolic acid | |
| Cannabinerol | |
| Carmagerol | |
| rac-6′-Epoxycannabigerol | |
| rac-6′-Epoxycannabigerolic acid | |
| rac-6′-Epoxycannabinerol | |
| rac-6′-Epoxycannabinerolic acid | |
| γ-Eudesmyl cannabigerolate | |
| γ-Cadinyl cannabigerolate | |
| Sesquicannabigerol | |
| Deprenyl O-methyl cannabigerolic acid (Amorfrutin 2) | |
| 5-Acetyl-4-hydroxycannabigerol | |
| Acetylcannabigeroquinol | |
| Cannabigeroquinone | |
| Abnormal cannabigerol | |
| Acetyl abnormal hydrocannabigeroquinol | |
| Abnormal cannabigeroquinol | |
| 2′-Hydroxy-1′,2′-dihydrocannabichromene (Cyclo-CBG) | |
| Cannabiorcichromene | |
| Cannabiorcichromenic acid | |
| Chlorcannabiorcichromenic acid | |
| Cannabivarichromene | |
| Cannabichromevarinic acid | |
| Cannabichromene | CBC |
| Cannabichromenic acid | |
| 4-Acetoxycannabichromene | |
| Anthopogochromenic acid | |
| Confluentin | |
| Daurichromenic acid | |
| 8′-Hydroxyisocannabichromene | |
| 4-Acetoxycannabichromene | |
| Cannabidiorcol | |
| Cannabidivarin | CBDV |
| Cannabidivarinic acid | |
| nor-Cannabidiol | |
| Cannabidiol | CBD |
| O-Methlycannabidiol | |
| O-Propylcannabidiol | |
| O-Pentylcannabidiol | |
| Cannabidiolic acid | CBDA |
| CBDA-THC ester | |
| Ferruginene C | |
| Cannabioxepane | |
| Cannabinodivarin | |
| Cannabinodiol | |
| Cannabifuran | |
| Dehydrocannabifuran | |
| trans-Δ-8-Tetrahydrocannabinol | |
| trans-Δ-8-Tetrahydrocannabinolic acid | |
| 10α-Hydroxy trans-Δ-8-tetrahydrocannabinol | |
| 10β-Hydroxy trans-Δ-8-tetrahydrocannabinol | |
| 11-Acetoxy-Δ-8-tetrahydrocannabinoic acid | |
| 10-Hydroxy-9-oxo-Δ-8-tetrahydrocannabinol | |
| Δ-9-trans-Tetrahydrocannabiorcol | |
| Δ-9-trans-Tetrahydrocannabiorcolic acid | |
| Δ-9-trans-Tetrahydrocannabivarin | |
| Δ-9-trans-Tetrahydrocannabivarinic acid | |
| Δ-9-trans-nor-Tetrahydrocannabinol | |
| Δ-9-trans-nor-Tetrahydrocannabinolic acid | |
| Δ-9-trans-Tetrahydrocannabinol | |
| Δ-9-trans-Tetrahydrocannabinolic acid A | |
| Δ-9-trans-Tetrahydrocannabinolic acid B | |
| 8α-Hydroxy-Δ-9-trans-tetrahydrocannabinol | |
| 8β-Hydroxy-Δ-9-trans-tetrahydrocannabinol | |
| 8-Oxo-Δ-9-trans-tetrahydrocannabinol | |
| O-Propyl-Δ-9-trans-tetrahydrocannabinol | |
| O-Penty1-Δ-9-trans-tetrahydrocannabinol | |
| 2-Formyl-Δ-9-trans-tetrahydrocannabinol | |
| β-Fenchyl Δ-9-trans-Tetrahydrocannabinolate | |
| α-Fenchyl Δ-9-trans-Tetrahydrocannabinolate | |
| Bornyl Δ-9-trans-Tetrahydrocannabinolate | |
| epi-Bornyl Δ-9-trans-Tetrahydrocannabinolate | |
| α-Terpinyl Δ-9-trans-Tetrahydrocannabinolate | |
| 4-Terpinyl Δ-9-trans-Tetrahydrocannabinolate | |
| γ-Eudesmyl Δ-9-trans-Tetrahydrocannabinolate | |
| α-Cadinyl Δ-9-trans-Tetrahydrocannabinolate | |
| Hexahydrocannabinol | |
| Hydroxy Δ-9,11-hexahydrocannabinol | |
| Methylen-bis Δ-9-trans-Tetrahydrocannabinol (Cannabisol) | |
| Tetrahydrocannabinol epoxide | |
| Δ-9-trans-Tetrahydrocannabinol glycol (cannabiripsol) | |
| 6a,7,10a-Trihydroxy-Δ-9-tetrahydrocannabinol | |
| Δ-9-cis-Tetrahydrocannabivarin | |
| Δ-9-cis-Tetrahydrocannabinol | |
| Cannabicitran (citrilidene-cannabis) | CBT |
| Cannabiorcicitran | |
| Bis-nor cannabitriol | |
| Bis-nor-Cannabitriol isomer | |
| 10-O-Ethyl bis-nor cannabitriol | |
| Isocannabitriol | |
| Cannabitriol | |
| Cannabitriol isomer | |
| 10-O-Ethyl cannabitriol isomer | |
| 10-Oxo-Δ6a(10a)-tetrahydrocannabinol | |
| 9,10-Anhydrocannabitirol | |
| Cannabiglendol | |
| 7,8-Dehydro-10-O-ethylcannabitriol | |
| Δ-7-cis-Isotetrahydrocannabivarin | |
| Δ-7-trans-Isotetrahydrocannabivarin | |
| Δ-7-trans-isotetrahydrocannabinol | |
| Bis-nor-cannabielsoin | |
| Bis-nor-Cannabielsoic acid B | |
| Cannabielsoin | CBE |
| Cannabielsoic acid A | |
| Cannabielsoic acid B | |
| Ferruginene A | |
| Ferruginene B | |
| Cannabiorcicyclol | |
| Cannabiorcicyclolic acid | |
| Cannabicyclovarin | |
| Cannabicyclol | CBL |
| Cannabicyclolic acid | |
| Anthopogocyclolic acid | |
| Rhododaurichromanic acid A | |
| Cannabiorcol | |
| nor-Cannabivarin | |
| Cannabivarin | CBV |
| nor-Cannabinol | |
| Cannabinol | CBN |
| Cannabinolic acid | |
| O-Methylcannabinol | |
| O-Propylcannabinol | |
| O-Penthylcannabinol | |
| 7-Hydroxcannabinol | |
| 8-Hydroxycannabinol | |
| 8-Hydroxcannabinolic acid | |
| 7,8-Dihydrocannabinol | |
| 4-Terpenyl cannabinolate | |
| Cannabicoumaronone | |
| Cannabicourmarononic acid | |
| Bisnor-Cannabichromanone | |
| Cannabichromanone | |
| (6aR)-Cannabichromanone B | |
| (6aR)-Cannabichromanone C | |
| Cannabichromanone | |
| Cannabimovone | |
| Anhydrocannabimovone | |
| Demethyldecarboxyamorfrutin A | |
| Tetrahydrocannabiphorol | THCP |
| Cannabidiphorol | CBDP |
| Cannabimovone | CBM |

Tetrahydrocannabinol, commonly referred to as “THC”, Isolate THC, or Full Spectrum THC, can include Tetrahydrocannabinol Delta-7, Tetrahydrocannabinol Delta-8, Tetrahydrocannabinol Delta-9, Tetrahydrocannabinol Delta-li, Tetrahydrocannabinol Delta-10, Tetrahydrocannabinol Delta-13, Tetrahydrocannabivarin (THCV) and Tetrahydrocannabinolic acid (THCA). In some instances, THC can comprise trans-THC, cis-THC or both. In some cases, THC can exist as a stereoisomer, such as, (+)-trans-THC; (−)-trans-THC; (+)-cis-THC and (−)-cis-THC. In some cases, cis-TCH can comprise, (+)-cis-THC, (−)-cis-THC, or both. In some cases, trans-THC can comprise (+)-trans-THC, (−)-trans-THC, or both. In some cases, a composition can comprise a ratio (weight to weight) of trans-THC to cis-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-THC to trans-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000.

In some cases, CBD can comprise trans-CBD. In some cases, trans-CBD can comprise (+)-trans-CBD, (−)-trans-CBD, or both. In some instances, CBD can comprise an enantiomer, or a diastereomer. In some instances, CBD can comprise a racemate. In some instances, CBD can comprise trans-CBD, cis-CBD or both. In some cases, CBD can comprise (1R,6R)-CBD, (1R,6S)-CBD, (1S,6R)-CBD, (1S,6S)-CBD, or a combination thereof. In some cases, a composition can comprise a ratio (weight to weight) of trans-CBD to cis-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of cis-CBD to trans-CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, CBD can be a powder, a liquid, an oil, an emulsion, an aerosol, a solid or a combination thereof. In some cases, CBD can be at least partially water soluble. In some instances, a cannabinoid can be a racemate. In some instances, a cannabinoid can comprise an isomer. In some instances, a cannabinoid can comprise an enantiomer, or a diastereomer.

In some cases, a composition can comprise a ratio (weight to weight) of THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, CBD can be mixed in a composition with Δ8-THC, Δ9-THC, Δ10-THC or a combination thereof. In some cases, a composition can comprise a ratio (weight to weight) of Δ8-THC, Δ9-THC, or Δ10-THC to CBD of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some cases, a composition can comprise a ratio (weight to weight) of CBD to Δ8-THC, Δ9-THC, or Δ10-THC of about: 1:1 to about 1:5, 1:4 to about 1:15, 1:10 to about 1:30, 1:20 to about 1:60, 1:40 to about 1:80, 1:75 to about 1:150, or about 1:100 to about 1:1000. In some instances, a cannabinoid or a salt thereof can be derived from hemp. In some instances, a cannabinoid or a salt thereof can be derived from cannabis. In some instances, a tetrahydrocannabinol or a salt thereof can be derived from hemp. In some instances, tetrahydrocannabinol Delta-8 or a salt thereof can be derived from hemp. In some instances, tetrahydrocannabinol Delta-8 or a salt thereof can be derived from cannabis. In some cases, a cannabinoid can be a synthetic cannabinoid or a salt thereof. In some cases, a cannabinoid can include a derivative of a cannabinoid or a salt thereof. In some instances, a cannabinoid can comprise an isomer of a cannabinoid. A derivative of a compound disclosed herein, can refer to a chemical substance related structurally to a compound disclosed herein. A derivative can be made from a structurally-related parent compound in one or more steps. In some cases, the general physical and chemical properties of a derivative can be similar to a parent compound.

In some embodiments, active pharmaceutical ingredients or salts thereof can comprise a THC, a CBD, a pharmaceutically acceptable salt of any of these, or any combination thereof. In some embodiments, active pharmaceutical ingredients can be THC or a pharmaceutically acceptable salt thereof.

The chemical formula for tetrahydrocannabinol is C₂₁H₃₀O₂ as shown in Formula (I). The chemical properties for tetrahydrocannabinol are described in Table 2.

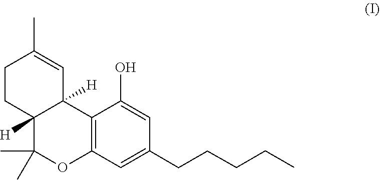 [SMILES: CCCCCc1cc(O)c2c(c1)OC(C)(C)[C@@H]1CCC(C)=C[C@@H]21]

Tetrahydrocannabinol (THC) is one a cannabinoid identified in cannabis. THC can be a psychoactive constituent of cannabis. The term THC can also refer to cannabinoid isomers, for example (−)-trans-Δ⁹-tetrahydrocannabinol. THC can be a lipid found in cannabis.

TABLE 2
Chemical and Physical Data of THC

| | |
|---|---|
| Formula | C₂₁H₃₀O₂ |
| Molar Mass | 314.469 g · mol⁻¹ |
| Specific Rotation | −152° (ethanol) |
| Boiling Point | 155-157° C. @ 0.05 mmHg, 157-160° C. @ |
| | 0.05 mmHg |
| Solubility in Water | 0.0028, (23° C.) mg/mL (20° C.) |

In some embodiments, an active pharmaceutical ingredient or salt thereof can be formulated as a powder. For example, a microencapsulated THC oil disclosed herein can be formulated as a powder using the methods described herein.

In some embodiments, a terpene, a flavonoid or both can be added to a composition described herein. In some instances, a terpene can comprise limonene, myrcene, pinene, caryophyllene, linalool, limonene, a salt of any of these or any combination thereof. In some cases, a flavonoid can comprise an anthocyanidin, an anthoxanthin, a flavanone, a flavanonol, a flavan, an isoflavonoid or any combination thereof. In some instances, a terpene, a flavonoid or both can be present in a composition described herein in an amount of about: 0.1%, 0.2%. 0.3%, 0.4%, 0.5%, 0.6%, 0.7%, 0.8%, 0.9%. 1%, 2%, 3%, 4%, 5%, 6%, 7%, 8%, 9%, or 10% of the total weight of the composition.

In some embodiments, the active pharmaceutical ingredients can comprise phosphodiesterase inhibitors or pharmaceutically acceptable salts thereof. In some embodiments, the phosphodiesterase inhibitors can be phosphodiesterase type 5 inhibitors (PDE5 inhibitors). In some embodiments, the phosphodiesterase type 5 inhibitors can include Sildenafil Citrate (Viagra), Tadalafil (Cialis) Avanafil (Stendra), and Vardenafil Hydrochloride (Levitra). In some cases, a PDE-V inhibitor can comprise sildenafil, tadalafil, avanafil, vardenafil, an ester thereof, a salt thereof, or any combination thereof. In some cases, a PDE-V inhibitor can comprise mirodenafil, udenafil, lodenafil, zaprinast, icariin, an ester of any of these, a pharmaceutically acceptable salt of any of these, or any combination thereof. In some cases, a PDE-V inhibitor can comprise lodenafil carbonate. In some cases, a phosphodiesterase inhibitor can comprise a selective phosphodiesterase inhibitor, a nonselective phosphodiesterase inhibitor, a PDE-I selective inhibitor, a PDE-II selective inhibitor (e.g. EHNA (erythro-9-(2-hydroxy-3-nonyl)adenine)), a PDE-III selective inhibitor, a PDE-IV selective inhibitor, a PDE-V selective inhibitor, a PDE-VI selective inhibitor, a PDE-VII selective inhibitor, a PDE-IX selective inhibitor, a PDE-X selective inhibitor, a PDE-XI selective inhibitor, a pharmaceutically acceptable salt of any of these, or any combination thereof. In some cases, an active pharmaceutical ingredient can comprise oxindole, inamrinone, anagrelide, cilostazol, mesembrenone, rolipram, ibudilast, roflumilast, apremilast, crisaborole, sildenafil, tadalafil, vardenafil, udenafil, avanafil, dipyridamole, quinazoline, paraxanthine, papaverine, a pharmaceutically acceptable salt of any of these, an ester of any of these, or any combination thereof. In some cases, a PDE5 inhibitor or a salt thereof such as sildenafil or a salt thereof can be administered in a composition comprising a cannabinoid described herein.

In some embodiments, active pharmaceutical ingredients or salts thereof can comprise an antibiotic, an antiviral, an antiparasitic, a diuretic, a blood pressure medication, a phosphodiesterase inhibitor, a pharmaceutically acceptable salt of any of these, or any combination thereof. In some cases, an active pharmaceutical ingredient can comprise a beta-blocker, an ACE inhibitor, an angiotensin II receptor blocker, a calcium channel blocker, an alpha blocker, an alpha-beta-blocker, a central agonist, a vasodilator, an aldosterone receptor antagonist, a cancer chemotherapeutic, a steroid, an immunomodulator, a pharmaceutically acceptable salt of any of these, or any combination thereof. In some cases, an antibiotic can comprise a penicillin, a cephalosporin, a tetracycline, an aminoglycoside, a macrolide, clindamycin, a sulfonamide, a trimethoprim, a metronidazole, a quinolone, or a nitrofurantoin. An antiviral can comprise an acyclovir, peramivir, zanamivir, oseltamivir phosphate, remdesivir, baloxavir marboxil, a salt of any of these or any combination thereof. In some cases, a beta-blocker can comprise an acebutolol, an atenolol, a betaxolol, a bisoprolol, a bisoprolol/hydrochlorothiazide, a metoprolol, a metoprolol tartrate, a metoprolol succinate, a nadolol, a pindolol, a propranolol, a sotalol, a timolol, a salt of any of these, or any combination thereof. In some cases, an ACE inhibitor can comprise a benazepril, a captopril, an enalapril, a fosinopril, a lisinopril, a moexipril, a perindopril, a quinapril, a ramipril, a trandolapril, a salt of any of these, or any combination thereof. In some cases, an angiotensin II receptor blocker can comprise a candesartan, an eprosartan, an irbesartan, a losartan, a telmisartan, a valsartan, a salt of any of these, or any combination thereof. In some cases, a calcium channel block can comprise an amlodipine, a diltiazem, a felodipine, an isradipine, a nicardipine, a nifedipine, a nisoldipine, a verapamil, a salt of any of these, or any combination thereof. In some cases, an alpha blocker can comprise a doxazosin, a prazosin, a terazosin, a salt of any of these, or any combination thereof. In some cases, an alpha-beta-blocker can comprise a carvedilol, a labetalol, a salt of any of these, or any combination thereof. In some cases, a central agonist can comprise a methyldopa, a clonidine, a guanfacine, a salt of any of these, or any combination thereof. In some cases, a vasodilator can comprise a hydralazine, a minoxidil, a salt of any of these, or any combination thereof. In some cases, an aldosterone receptor antagonist can comprise an eplerenone, a spironolactone, a salt of any of these, or any combination thereof. In some cases, an active ingredient can comprise a direct renin inhibitor, for example an aliskiren or a salt thereof.

In some embodiments, the composition can further comprise: another set of active pharmaceutical ingredients or salts thereof. For example, a second, third, or fourth different set of active pharmaceutical ingredients. In some embodiments, the additional pharmaceutical ingredients or salts thereof can be administered in parallel or consecutively to enhance the efficacy of the first set of active pharmaceutical ingredients or salts.

In some embodiments, a composition can further comprise: an additional set of active pharmaceutical ingredients or salts thereof which can be administered in parallel or consecutively to enhance the efficacy of cannabinoids or salt thereof. In some embodiments, the second different set of active pharmaceutical ingredients or salts administered in parallel or consecutively to THC can be PDE5 inhibitors. In some embodiments, a composition can comprise two or more different sets of active pharmaceutical ingredients or salt thereof which can be administered in parallel or consecutively to enhance the efficacy of cannabinoids or salt thereof. For example, a composition can comprise two or more cannabinoids such as THC and CBD.

In some embodiments the first set of active ingredients or salts can be administered in parallel or consecutively with a second different set of active ingredients. In some cases, the active ingredients can comprise nitrates, nitric oxide, nitric oxide generating components, nitrite salts, nitrate salts, sodium nitrates, potassium nitrates, vitamin C, ascorbic acid, L-arginine, L-citrulline, vitamin B12, magnesium ascorbate, sodium ascorbate, potassium ascorbate, antihypertensive agents, diuretics, salts thereof, or any combination thereof. In some cases, the pharmaceutical ingredients can comprise beta blockers (β-blockers), calcium blockers, angiotensin converting enzyme inhibitors, angiotensin receptor blockers, Nebivolol, CYP3A4 inhibitors, ketoconazole (Nizoral), itraconazole (Sporanox), erythromycin, saquinavir, clarithromycin, HIV protease inhibitors, alpha-adrenergic blocking agents (α-blockers), salts thereof, or any combination thereof.

In some embodiments, a second different set of active pharmaceutical ingredients or salts may not be comprised in a composition. In some embodiments, a second different set of active pharmaceutical ingredients or salts not comprised in a composition can be administered concurrently, in parallel, or consecutively.

In some embodiments, the pharmaceutical composition has metabolites that can be pharmacologically active, retaining, at least partially, the potency of the parent drug or the parent pharmaceutical component.

In some embodiments, the composition comprising the salt of the pharmaceutically active ingredient, wherein the salt comprises an organic salt, an inorganic salt, or any combination thereof. In some cases, an organic salt can comprise a phosphinate (e.g. sodium hypophosphite), a hydrazinium salt, a urate, a diazonium salt, an oxalate salt, a tartrate, a choline chloride. An example of an inorganic salt can be sodium chloride, calcium chloride, magnesium chloride, sodium bicarbonate, potassium chloride, sodium sulfate, calcium carbonate, calcium phosphate, or any combination thereof.

In some embodiments, the composition comprising the salt of the pharmaceutically active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a palmitic acid salt, a phosphoric acid salt, or any combination thereof.

In some embodiments, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

### Excipients and Other Non-Active Ingredients

In some embodiments, the composition can comprise a pharmaceutically acceptable: excipient, carrier, and/or diluent. In some embodiments, the composition comprises pharmaceutically acceptable excipients. In some cases, a pharmaceutically acceptable excipient can comprise an excipient. As used herein, “excipient” can refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some embodiments, an excipient can comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO Procipient®, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, a pharmaceutically acceptable excipient can comprise a carbohydrate. In some instances, the carbohydrate can comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. In some embodiments, a pharmaceutically acceptable excipient can comprise lactose. In some instances, lactose can comprise milled lactose, sieved lactose, micronized lactose, spray dried lactose, anhydrous lactose, monohydrate lactose, or a combination thereof. In some cases, an excipient can comprise a fumaryl diketopiperazine (FDKP). In some cases, an excipient can comprise a 1,2-distearoyl-sn-glycero-3-phosphocholine.

In some embodiments, the active ingredient or pharmaceutically acceptable salt thereof can be contained at least in part within an excipient. In some embodiments, the active ingredient or pharmaceutically acceptable salt thereof can be contained at least in part in an excipient. In some embodiments, the active ingredient can be contained within a pore of an excipient. The “pore” of the excipient can refer to excipient particles that have been engineered to have open or closed pore structures. Porous excipient particles may be carriers of pharmaceutically active ingredients. Porous excipient particles may have a large surface area, stable structure, adjustable pore sizes, tunable dissolution, diffusion, or distribution, and well-defined surface properties. Porous excipient particles may facilitate sustained-release unit doses.

In some embodiments, in addition to the active pharmaceutical ingredients or salts thereof, the compositions can further comprise inactive ingredients selected from the group consisting of microcrystalline cellulose, anhydrous dibasic calcium phosphate, croscarmellose sodium, magnesium stearate, hypromellose, titanium dioxide, lactose, triacetin, mannitol, xylitol, sorbitol, sugar alcohols, cellulose, cellulose esters, cellulose ethers, modified celluloses, starch, modified starches, polysaccharides, oligosaccharides, disaccharides, saccharides, gelatin, polyvinylpyrrolidone, polyethylene glycol, binders, flavorants, colorants, FD & C Blue #2 aluminum lake, magnesium stearate, antiadherent agents, stearate salts, sweeteners, silica, lubricants, or any combination thereof.

### Method of Making Compositions

In some cases, methods of making a composition can comprise creating particles by the methods described herein. In some cases, particles can comprise an excipient (e.g. a pharmaceutically acceptable excipient), an active ingredient, or both. In some embodiments, a method of making a powdery composition, can comprise mixing, in a mixer, particles of a pharmaceutically acceptable excipient; and particles comprising an active ingredient or a pharmaceutically acceptable salt thereof at least partially encapsulated in two or more coating materials. In some cases, the particles at least partially encapsulated in two or more coating materials are spray dried or milled.

In some cases, a composition can comprise a mixture of particles described herein. In some instances, at least a portion of the particles of the a pharmaceutically acceptable excipient can have a particle diameter ranging from about 50 micrometers to about 200 micrometers, as measured by a particle size analyzer using laser diffraction; at least a portion of the particles comprising the active ingredient, or the pharmaceutically acceptable salt thereof encapsulated in the coating material can have a particle diameter ranging from about 20 micrometers to about 200 micrometers, or from about 40 micrometer to about 70 micrometers, as measured by a particle analyzer using laser diffraction; and wherein in a human clinical trial, the powdery composition, when taken orally, can provide in at least part of the humans in the clinical trial a time to peak plasma concentration (Tmax) of the active ingredient or the salt thereof ranging from about 15 minutes to about 90 minutes, or from about 15 minute to about 45 minutes.

In some embodiments, a method of making the powdery composition can comprise the following steps: microencapsulation of active pharmaceutical ingredient; spray drying, atomization and dry powder collection, applying an enteric coating in a fluid bed, blending of active pharmaceutical ingredient with excipient.

In some cases, a method of making a composition herein can comprise contacting a composition disclosed herein with a liquid, an excipient, a carrier, a diluent, a food, a medical food, a capsule, a vitamin, an unsaturated fatty acid, an ester thereof, a salt thereof, an organic salt, an inorganic salt, or any combination thereof.

### Microencapsulation

In some embodiments, encapsulation can comprise microencapsulation.

Microencapsulation can be a process in which a microcapsule can be created as a small sphere or multi-sphere with a core and a matrix wall around it. The pharmaceutical ingredient inside the microcapsule can be called a fill. In some cases, a fill can be a liquid, an oil, a solid or any combination thereof. In some cases, a fill can comprise a previously microencapsulated particle. The wall around the fill (“or core”) can be referred to as a shell, a coating, or a membrane. In some cases, a microcapsule can comprise more than one shell. For example, a microcapsule can comprise 2, 3, 4, 5, 6, 7, 8, 9, or more shells. In some cases, a microcapsule can have a diameter of about 20 microns in size. In some instances, microcapsules can have a diameter as small as 20 micron in size to about 200 microns in size or about 40 micron to about 70 microns in size. In some cases, the small size can provide a pharmaceutical ingredient a large surface area. In some cases, the small size can provide a pharmaceutical ingredient a large surface area to be available for absorption, release, transfer, or any combination thereof. In some cases, microencapsulation can increase the solubility of an active ingredient, for example a microencapsulated cannabinoid oil can have increased solubility compared to an unencapsulated cannabinoid oil. In some instances, crystal geometry and structure can be controlled by the spray drying process. Microencapsulation can generate crystals with amorphous structure. In some instances, an amorphous crystal can lack sharp edges and angles, which may cause irritation. In some cases, an amorphous crystal can have a rounded edge. In some instances, an amorphous crystal may have increased bioavailability.

In some instances, a composition in an oil formulation can be microencapsulated with compatible diluents to protect the oil from oxidation and provide a longer shelf life than the unprotected composition. The diluents can be aqueous, or solvent based and use animal or plant materials. In some cases, the diluent can comprise alcohols: e.g., ethanol, butanol, 2-ethylhexanol, isobutanol, isopropanol, methanol, propanol, propylene glycol; ketones: e.g., acetone, methyl ethyl ketone, methyl isobutyl ketone, methyl isopropyl ketone, mesityl oxide, trichloroethylene; halogenated solvents: e.g., ethylene bromide, chloroform, ethylene chloride, dichloromethane, tetrachloroethylene, carbon tetrachloride; amides: e.g., dimethylformamide; ethers: e.g., 1,4-dioxane, butyl ether, ethyl ether, di-isopropyl ether, tetrahydrofuran, tert-butyl methyl ether; sulfur containing solvent: e.g., dimethyl sulfoxide; amines: e.g., pyridine; nitriles: e.g., acetonitrile; esters: e.g., ethyl acetate; aliphatic hydrocarbons: e.g., cyclohexane hexane; aromatic hydrocarbons: e.g., toluene xylene; water or any combinations thereof. In some cases, the diluent can comprise benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, n-methylpyrrolidone, nitromethane, pyridine, sulfolane, tetralin, toluene, 1,1,2-trichloroethylene, xylene or any combinations thereof.

In some embodiments, a particle that has previously been microencapsulated can be microencapsulated with another coating. For example, an initial coating can be applied to cover the fill (e.g., the active ingredient) then a second coating can be applied to the first coating. In some cases, a third coating can be applied to the second coating. In some cases, a fourth coating can be applied to the third coating. In some cases, a fifth coating can be applied to a fourth coating. In some cases, a sixth coating can be applied to the fifth coating. In some cases, a seventh coating can be applied to a sixth coating. In some cases, an eighth coating can be applied to the seventh coating. In some cases, a ninth coating can be applied to the eighth coating. In some cases, additional coatings can be applied to the ninth coating.

In some embodiments, after each microencapsulation process, the suspension can be spray dried to create a dry powder product. In some embodiments, after a particle has been microencapsulated, an addition coating, for example, an enteric coating, may be applied by a fluidized bed system.

### Spray Drying Atomization and Dry Powder Collection

In some embodiments, a method of making the powdery composition can comprise particles wherein at least a portion of the particles of the active ingredient or a pharmaceutically acceptable salt thereof can be made by a spray drying process.

In some embodiments, the spray drying process can comprise: atomizing liquid droplets comprising the active ingredient or the pharmaceutically acceptable salt thereof, drying the droplets from particles, recovering the particles, or any combination thereof. In some cases, the liquid droplets can comprise an encapsulated active ingredient.

In some embodiments, the spray drying process can comprise: atomizing liquid droplets comprising a microencapsulated active ingredient or the pharmaceutically acceptable salt thereof, drying the droplets from particles, recovering the particles, or any combination thereof. In some cases, the liquid droplets can comprise an encapsulated active ingredient with two or more coatings.

In some embodiments, a spray drying manufacturing system can comprise a closed spray dryer container which receives the solution comprising a drug dissolved in a suitable solvent (aqueous or solvent based). In some cases, a solvent can comprise alcohol, ethanol, dimethylformamide (DMF), dimethyl sulfoxide (DMSO), a polar organic solvent, an organic solvent, or any combination thereof. In some embodiments, a spray drying manufacturing system can comprise a closed spray dryer container which receives a solution comprising a microencapsulated drug dissolved or dispersed in a suitable solvent (aqueous or solvent based). In some embodiments, the solution then enters the particle formation chamber which can be connected to an atomizer located at the top of the chamber. In some cases, the atomizer can use a gas. In some embodiments, the atomizer can be a two component or rotary nozzle type that distributes the solution into fine droplets controlled by the atomizer pressure. In some embodiments, the atomizer can be a rotary atomizer that employ an atomizer wheel rotating at high speed.

In some embodiments, this atomization gas can be an inert gas. As used herein, “inert gas” can refer to a non-reactive gas, or a gas that does not undergo chemical reactions under a set of given conditions. Inert gases can be generally used to avoid unwanted chemical reactions degrading a sample, or to prevent bacterial growth. These undesirable chemical reactions can often be oxidation and hydrolysis reactions with the oxygen and moisture in air. The term “inert gas” can be context-dependent because several of the noble gases, which have been historically referred to as the inert gases, can be made to react under certain conditions. In some embodiments, inert gas can be air, nitrogen, carbon dioxide or any combination thereof. In some embodiments, the atomized droplets go through a hot gas drying chamber to produce uniform fine particles that maintain a tight particle size distribution following liquid evaporation. In some cases, the solid particle forms and falls to the bottom of the drying chamber. In some instances, the balance between temperature, flow rate, and droplet size can control the drying process. In some embodiments, the powder can be recovered from the exhaust gas using a cyclone or a bag filter.

The moisture level of the powder after spray drying can be below about 10%. In some embodiments, the moisture level can be below about 15%, about 14%, about 13%, about 12%, about 11%, about 10%, about 9%, about 8%, about 7%, about 6%, about 5%, about 4%, about 3%, about 2%, or about 1%.

### Additional Coatings (e.g., an Enteric Coating) of the Microencapsulated Powder

In some embodiments, an addition coating, for example, an enteric coating, may be applied by a fluidized bed system or by a rotary nozzle spray dry system. In some embodiments the microencapsulated powder can be processed through a fluidized bed system to coat the particles with an enteric coating. This can enable a pH dependent release of the active pharmaceutical ingredient. In some embodiments, an encapsulated powder can be processed through a fluidized bed and coated with an additional enteric coating material that can be configured to allow the dry powder to bypass the stomach and release the powder in the intestines. One or more additional coatings on microencapsulated particles can be created by spraying a polymer binder as a solution, suspension or melt onto a fluidized powder particle bed. In some embodiments, after the initial spray dry encapsulation and subsequent fluidized bed coating, the particles can be blended in a V-type blender with an appropriate excipient. The excipient can help the powder maintain flow and prevent clumping.

In some cases, a fluidized bed system can coat microencapsulated particles with an enteric, polymer coating. For example, the system can apply an additional coating, such as an enteric coating to the spray dried encapsulated powder.

Blending of Active Pharmaceutical Ingredient with Excipient

In some embodiments, a particle size can be validated by a Malvern particle analyzer prior to blending with an excipient carrier. In some embodiments, the active powder (e.g. the powdery composition) can be blended with an excipient carrier (lactose) product in a Patterson Kelly (PK Blender) and the blended powder can be fed to a hopper.

### Encapsulation

The core active ingredient o can be microencapsulated with an amphipathic molecule that has both a polar end (“hydrophilic”) and non-polar end (“hydrophobic”). In some cases, a hydrophilic end of an amphipathic molecule may interact with core material. In some cases, a hydrophobic end of an amphipathic molecule may interact with core material. This hydrophilic and hydrophobic structure can enable the molecule to microencapsulate an active ingredient or a previously microencapsulated active ingredient and form a microsphere. In some instances, the microencapsulated particle may have a hydrophilic exterior and a hydrophobic interior. In some instances, the microencapsulated particle may have a hydrophobic exterior and a hydrophilic interior. In some cases, the microencapsulation process can coat the active ingredient, which is the core, by the amphipathic encapsulating agent, which is the wall material, so that the active ingredient is at least partially surrounded by a wall of the amphipathic material. For example, hydroxypropyl methylcellulose acetate succinate (HPMCAS) can be an amphipathic molecule used to coat an oil comprising a cannabinoid or a salt thereof. The microencapsulation blend can be a spray dried dispersion, that can be fed into a spray dry system to create a hard-outer coating on the microcapsules.

The wall material for microencapsulation and the enteric coating can form a film that is cohesive with the core active ingredient or with a previously applied wall material. A wide variety of coating materials are available for encapsulation, e.g., traditional coating materials like inert polymers and pH sensitive ones as carboxylate and amino derivatives, which swell or dissolve according to the degree of cross-linking; some innovative coating polymers have also been developed for applications particularly among the bioadhesives and mucoadhesives. In some cases, the coating material can be hydrophilic polymers, hydrophobic polymers or a combination of both. In some cases, a microcapsule shell can comprise an amphipathic molecule. In some cases, the coating material can be gelatin, polyvinyl alcohol, ethyl cellulose, cellulose acetate phthalate and styrene maleic anhydride. In some instances, the coating material may not react with the pharmaceutical ingredient. In some instances, the coating material may not react with a previously applied coating (e.g., wall material). In some cases, a microcapsule shell can comprise Hydroxypropyl methylcellulose (“HPMC”), Hydroxypropyl methylcellulose Acetate Succinate (“HPMCAS”), a cyclodextrin, maltodextrin, povidone, copovidone and others. In some instances, a microcapsule shell can comprise HPMCAS-LG, HPMCAS-MG, HPMCAS-HG or HPMC-P or a combination thereof. In some instances, a microcapsule shell can comprise a different grade of HPMC or HPMCAS. For example, a microcapsule shell can comprise an E5, an E50, or a K4M grade of HPMC. In another example, a microcapsule shell can comprise a L, a M, or an H grade of HPMCAS. In some cases, a microcapsule shell can comprise a HPMCAS. In some cases, a microcapsule shell can comprise gelatin, cornstarch, polyvinylpyrrolidone (PVP), an oligosaccharide, a long chain sugar or any combination thereof. In some cases, PVP can comprise PVP0, PVP1, PVP2, PVP3, PVP4, PVP5, PVP6, PVP7, or any combination thereof. In some cases, a microcapsule shell may comprise a FDKP (fumaryl diketopiperazine). In some cases, a microcapsule shell can comprise a 1,2-distearoyl-sn-glycero-3-phosphocholine. In some cases, a microcapsule shell may comprise a FDKP and a 1,2-distearoyl-sn-glycero-3-phosphocholine. In some cases, a microcapsule shell can comprise a fatty acid, a liposome, an amino acid, a natural oil and a sugars, trehalose, dextran, a natural oil, a synthetic oil or a combination thereof. In some instances, an amino acid can comprise glutamic acid, aspartic acid, lysine, tryptophan, tyrosine, methionine or a combination thereof. In some cases, a coating material may not comprise diketopiperazine, leucine, trehalose, distearoylphosphatidylcholine (DSPC) or a combination thereof. In some cases, a fatty acid can comprise a polyunsaturated fatty acid, an essential fatty acid, a conjugated fatty acid, a short chain fatty acid, a medium chain fatty acid, a long chain fatty acid, a very long chain fatty acid, a saturated fatty acid, an unsaturated fatty acid, a monounsaturated fat, or any combination thereof. In some cases, a fatty acid can comprise an omega-3, an omega-5 fatty acid, an omega-6, an omega-7 fatty acid, an omega-9 fatty acid, an omega-10 fatty acid, an omega-11 fatty acid, an omega-12 fatty acid, or a combination thereof. In some cases, a natural oil can comprise soybean oil, a vegetable oil, a food oil, evening primrose oil, borage oil, blackcurrant seed oil, flax or linseed oil, rapeseed or canola oil, corn oil, almond oil, avocado oil, Brazil nut oil, canola oil, cashew oil, chia seed oil, cocoa butter oil, coconut oil, corn oil, cottonseed oil, flaxseed/linseed oil, grape seed oil, hemp seed oil, vigna mungo oil, mustard oil, olive oil, palm oil, peanut oil, pecan oil, perilla oil, rice bran oil, safflower oil, sesame oil, soybean oil, walnut oil, sunflower oil, cottonseed oil, palm oil, or a combination thereof. In some cases, a microcapsule coating can comprise a polyvinylpyrrolidone (PVP), a povidone, a PVA (polyvinyl alcohol), Eudragit L30D, L-100, S, a liposome, a fatty acid, a long chain triglyceride, or any combination thereof. In some cases, a microcapsule shell can increase or decrease active ingredient release kinetics. In some cases, a microcapsule shell can increase or decrease bioavailability. In some cases, microencapsulation of a cannabinoid or a salt thereof can produce about: 5% to about 70%, 5% to about 10%, 5% to about 20%, 10% to about 30%, 15% to about 40%, 25% to about 40%, 10% to about 60%, or 20% to about 50% more bioavailability of the cannabinoid or the salt thereof as compared to the cannabinoid or the salt thereof that is not encapsulated. The wall material can be biodegradable and biocompatible with the pharmaceutical ingredient. In some cases, a microcapsule can be produced by dissolving or mixing the pharmaceutical ingredient in a solvent containing the shell material to produce a liquid suspension. For example, HPMCAS can be dissolved with ethanol and water and a pharmaceutical compound (e.g. the core) can be added the liquid suspension. In some instances, the pharmaceutical compound may not dissolve in the liquid suspension. In some instances, the pharmaceutical compound may dissolve in the liquid suspension. The liquid suspension can be dried with a spray drying technique described herein or by another method.

In some embodiments, a polymer coating an or an enteric coating comprises a barrier, such as a polymer barrier, that can be applied to a composition (for example a microencapsulated particle) to prevent dissolution or disintegration in the stomach. In some cases, this can enable the active ingredient to bypass the stomach to the small intestines before the active ingredient is released. In some cases, a wall material such as an additional coating on a previously microencapsulated particle can comprise an enteric coating. In some cases, an enteric coating can comprise a polymer. In some cases, an enteric coating can comprise methyl methacrylate (MMA). In some cases, an enteric coating can comprise a plant fiber, a shellac, a wax, a fatty acid, a plastic, or a combination thereof. In some cases, an enteric coating can comprise a methyl acrylate-methacrylic acid copolymer, a cellulose acetate phthalate (CAP), a cellulose acetate succinate, hydroxypropyl methyl cellulose phthalate, a HPMC-AS), a polyvinyl acetate phthalate (PVAP), a methyl methacrylate-methacrylic acid copolymer, a shellac, a cellulose acetate trimellitate, a sodium alginate, a zein, an enteric coating solution (an ethylcellulose, a medium chain triglycerides, an oleic acid, a sodium alginate, a stearic acid), or a combination thereof.

In some instances, a wall material coating can comprise an enteric coating, a time release coating, a pH dependent coating, a delayed release coating, an extended release coating, or a combination thereof. For example, one or more layers of an enteric coating can be added to an active ingredient by a microencapsulation process and/or fluidized system described herein to prevent it from dissolving until after it passes through the stomach. In some cases, two or more enteric coatings can be applied to an active ingredient. In some cases, a microencapsulated particle coating can release an active ingredient depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH microencapsulated particle coating can be used to dose areas of the GI tract with specific pH levels. In some embodiments, an enteric coating of a microencapsulated particle can be a polymer barrier that can be applied to the microencapsulated particle described herein to enable a controlled release. Bypassing the stomach can allow for more precise dosing and can enable the drug to achieve a higher bioavailability in the gastric tract. In some cases, these coatings or multiple layers of these coatings can be modified to deliver medicine from the mouth, all the way to the colon. In some cases, the technology can be applied to different microencapsulated layers of an active ingredient particle and utilize time-released, pH-controlled released, or a combination of both technologies to achieve the intended drug delivery. In some cases, one or more layers of a microcapsule shell can increase or decrease active ingredient release kinetics. In some cases, one or more layers of a microcapsule shell can increase or decrease bioavailability. In some cases, microencapsulation of a cannabinoid or a salt thereof can produce about: 5% to about 70%, 5% to about 10%, 5% to about 20%, 10% to about 30%, 15% to about 40%, 25% to about 40%, 10% to about 60%, or 20% to about 50% more bioavailability of the cannabinoid or the salt thereof as compared to the cannabinoid or the salt thereof that is not encapsulated when ingested by a subject.

In some embodiments, a microencapsulated particle can be configured (for example, with one or more layers of an enteric coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

The wall material can be biodegradable and biocompatible with the pharmaceutical ingredient. In some cases, the wall material can be biodegradable and biocompatible with a previously applied wall material. In some cases, a microcapsule can be produced by dissolving, dispersing, or mixing the pharmaceutical ingredient in a solvent containing the shell material to produce a liquid suspension. In some cases, a multiple coated microcapsule can be produced by dissolving, dispersing, or mixing a previously microencapsulated pharmaceutical ingredient in a solvent containing a second shell material to produce a liquid suspension. For example, HPMCAS can be dissolved with ethanol and water and a pharmaceutical compound (e.g. the core) can be added the liquid suspension. In another example, an active ingredient encapsulated by HPMCAS can be dispersed with water and an enteric shell can be added to the liquid suspension. In some instances, the pharmaceutical compound may not dissolve in the liquid suspension. In some instances, the pharmaceutical compound may dissolve in the liquid suspension. In some instances, an encapsulated particle may not dissolve in the liquid suspension. In some instances, an encapsulated particle may dissolve in the liquid suspension. A liquid suspension can be dried with a spray drying technique described herein or by another method.

In some cases, the average wall thickness can of an individual coating of a microencapsulated particle can be about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness can of an individual coating of a microencapsulated particle can range from about: 1 μm to about 10 μm, 1 μm to about 5 μm, 2 μm to about 7 μm, 3 μm to about 8 μm, 5 μm to about 10 μm, 5 μm to about 15 μm, or 1 μm to about 30 m. In some instances, the wall thickness of a microencapsulated particle can increase by increasing the ratio of the wall material to the core material prior to spray drying. In some cases, the ratio of wall material to core material (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material to core material (weight/weight) can be about 10:1. In some instances, the wall thickness of a microencapsulated particle can increase by increasing the ratio of the wall material to a previously microencapsulated particle prior to spray drying. In some cases, the ratio of wall material to previously microencapsulated particle (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material previously microencapsulated particle (weight/weight) can be about 10:1.

In some embodiments, in a plurality of microencapsulated particles about: 1%, 5%, 10%, 15%, 20%, 25%, 30%, 35%, 40%, 45%, 50%, 55% 60%, 65%, 70%, 75%, 80%, 90%, 95%, 99% or 100% of the microencapsulated particles can comprise a core substantially encapsulated by a wall material. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles can comprise a core substantially encapsulated by a wall material. In some cases, in a plurality of microencapsulated particles not all of the core material can be encapsulated by the wall material.

In some embodiments, in a plurality of microencapsulated particles with two or more coatings about: 1%, 5%, 10%, 15%, 20%, 25%, 30%, 35%, 40%, 45%, 50%, 55%, 60%, 65%, 70%, 75%, 80%, 90%, 95%, 99% or 100% of the microencapsulated particles can comprise two or more coatings. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90% to about 100% of the microencapsulated particles can comprise two or more coatings. In some cases, in a plurality of microencapsulated particles with two or more coatings not all of the microencapsulated particles can be encapsulated by the two or more coatings.

In some embodiments, microencapsulated particles or unencapsulated particles have a mean, a median, or a mode particle diameter of less than about: 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles or unencapsulated particles have a mean, a median, or a mode particle diameter of equal to, or more than about: 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles or unencapsulated particles have a mean, a median, or a mode particle diameter ranging from about: 20 μm to about 200 μm, 40 μm to about 70 μm, 30 μm to about 100 μm, 50 μm to about 150 μm, 60 μm to about 130 μm, 70 μm to about 160 μm, or 100 μm to about 200 μm.

The core material can be the material over which coating has to be applied to serve the specific purpose. Core material may be in form of solids or droplets of liquids and dispersions. In some cases, the core material can comprise a cannabinoid. In some cases, the core material can comprise an individual cannabinoid of two or more cannabinoids. In some instances, a cannabinoid can be an oil. The composition of core material can vary and thus furnish definite flexibility and allow effectual design and development of the desired microcapsule properties. A substance may be microencapsulated or microencapsulated with two or more coatings for a number of reasons. Examples may include protection of reactive material from their environment, safe and convenient handling of the materials which can be otherwise toxic or noxious, taste masking, means for controlled or modified release properties means of handling liquids as solids, preparation of free flow powders and in modification of physical properties of the drug. For example, encapsulation can improve solubility and dissolution and therefore increase bioavailability of an active ingredient such as a cannabinoid. Microencapsulation with two or more layers can provide solubility benefits, modified release properties, taste masking benefits, composition stability benefits or any combination thereof. Microencapsulation can be used to increase the stability, improve the handling properties of compounds, facilitate higher bioavailability when reconstituted or administered or any combination thereof.

In some instances, the core diameter of a microencapsulated particle can be about: 100 nm (nanometer), 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the core diameter of a microencapsulated particle can range from about: 100 nm to about 250 nm, 100 nm to about 500 nm, 100 nm to about 1 μm, 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the core can comprise about: 1%, 5%, 10%, 15%, 20%, 25%, 30%, 35%, 40%, 45%, 50%, 55%, 60%, 65%, 70%, 75%, 80%, 90%, 95% or 99% of the total microcapsule content (e.g. total weight of the core and wall material). In some instances, the core can comprise about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, or 1% to about 99% of the total microcapsule content.

In some cases, microencapsulation of a cannabinoid or a salt thereof by HPMCAS and/or an enteric coating can provide increased bioavailability by absorption in the intestines. For example, THC may not be water soluble where much of the active ingredient can be neutralized, however an enteric coating of the microencapsulation powder can provide increased absorption into the blood stream from the intestines. In some instances, enteric coating of the microencapsulation can increase the solubility of an active ingredient. In some cases, an enteric coated, microencapsulated cannabinoid or a salt thereof may be absorbed about: 10% to about 70%, 5% to about 10%, 5% to about 20%, 10% to about 30%, 15% to about 40%, 25% to about 40%, 35% to about 50%, 10% to about 60%, 40% to about 90%, or 20% to about 50% faster than a cannabinoid that is not microencapsulated and enteric coated. In some cases, a microencapsulated cannabinoid or a salt thereof may be absorbed after oral administration into the blood stream in about: 10 minutes to about 100 minutes, 15 minutes to about 90 minutes, 30 minutes to about 120 minutes, or 15 minutes to about 45 minutes. In some cases, a microencapsulated cannabinoid (or other active ingredient) or a salt thereof may be absorbed after oral administration into the blood stream in more than about, less than about, or equal to about: 10 minutes (min), 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 61 min, 62 min, 63 min, 64 min, 65 min, 66 min, 67 min, 68 min, 69 min, 70 min, 71 min, 72 min, 73 min, 74 min, 75 min, 76 min, 77 min, 78 min, 79 min, 80 min, 81 min, 82 min, 83 min, 84 min, 85 min, 86 min, 87 min, 88 min, 89 min, 90 min, 100 min, 110 min, 120 min, 150 min, or 180 min.

In some cases, a microencapsulated particle can have 1, 2, 3, 4, 5, 6 or more than 6 shell layers. In some cases, individual shell layers can comprise different compounds. In some cases, individual shell layers can comprise the same compound. In some cases, the different layers comprise the same compound. In some cases, the different layers comprise different compounds.

In some embodiments, a method of microencapsulation can comprise at least partially dissolving the coating material (e.g. HPMC or HPMCAS) in a solvent such as an ethanol and water mix. In some cases, a cannabinoid oil or salt thereof can be micronized with a micronizer to generate small oil droplets. In some cases, a microfluidic system can be used to generate small oil droplets. The oil droplets can be added to the solution of the coating material and the solvent to create a suspension of the oil droplets and the coating material dissolved in the solvent. In some instances, the oil droplets may not dissolve in the suspension and may remain in suspension. The suspension can be mixed to an at least partially uniform mixture and spray dried. The coating can at least partially encapsulate the oil droplets containing the cannabinoid or salt thereof. In some cases, the encapsulation of a cannabinoid can be a spherical, round, oval, or any shape structure. After the first encapsulation layer is added, additional layers can be added using methods described herein.

### Methods of Making the Powdery Composition to Select Sizes

In some embodiments, a method of making the powdery composition can comprise mixing particles of a pharmaceutically acceptable excipient and particles comprising an active ingredient or a pharmaceutically acceptable salt thereof at least partially encapsulated in two or more layers of a coating material. In some cases, the particles at least partially encapsulated in the two or more layers coating material are spray dried and/or encapsulated with a fluidized bed. In some embodiments, a method of making the powdery composition can comprise mixing particles in a mixer.

In some embodiments, the method of making the powdery composition can comprise mixing the particles described herein. In some instances, at least a portion of the particles of a pharmaceutically acceptable excipient can have a particle diameter ranging from about 50 micrometers to about 200 micrometers, as measured by a particle size analyzer using laser diffraction; and particles comprising an active ingredient or a pharmaceutically acceptable salt thereof at least partially encapsulated in a coating material and enteric coated can have a particle diameter ranging from about 20 micrometers to about 200 micrometers, or about 40 micrometers to about 70 micrometers, as measured by a particle size analyzer using laser diffraction; and wherein in a human clinical trial, the powdery composition, when taken orally, can provide in at least part of the humans in the clinical trial a time to peak plasma concentration (Tmax) of the active ingredient or the salt thereof ranging from about 15 minutes to about 90 minutes, or from about 15 minutes to about 45 minutes.

In some embodiments, at least a portion of the particles of a pharmaceutically acceptable excipient can have a particle diameter ranging from about: 30 μm (micrometers) to about 60 μm, 50 μm, to about 200 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, particles of a pharmaceutically acceptable excipient can have a particle diameter of more than about or equal to about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, particles of a pharmaceutically acceptable excipient can have a particle diameter of less than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, the particles of a pharmaceutically acceptable excipient can range from about 50 μm to about 100 μm, which may be preferred for oral administration. In some instances, particle size as can comprise the diameter, the radius, or length of a particle. In some instances, particle size can be a measure of the mean, the median or the mode of a plurality of particles.

In some embodiments, particles comprising an active ingredient or a pharmaceutically acceptable salt thereof at least partially encapsulated in two or more coating layers can have particle diameters ranging from about: 20 μm to about 200 μm, 40 μm to about 70 μm, 30 μm to about 100 μm, 50 μm to about 150 μm, 60 μm to about 130 μm, 70 μm to about 160 μm, or 100 μm to about 200 μm. In some cases, particles comprising an active ingredient or a pharmaceutically acceptable salt thereof at least partially encapsulated in two or more coating layers can have a particle diameter of less than about: 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some cases, particles comprising an active ingredient or a pharmaceutically acceptable salt thereof at least partially encapsulated in two or more coating layers can have a particle diameter of more than about or equal to about: 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some cases, particles comprising an active ingredient or a pharmaceutically acceptable salt thereof at least partially encapsulated in a coating material and in an enteric coating can have a particle diameter of about 40 μm to about 70 μm, which can be preferred when taken orally as an ingredient for absorption into intestines

In some embodiments, a particles or compositions described herein can have a tap density of more than about, less than about, or equal to: 0.1 grams/centimeter³ (g/cm³), 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some embodiments, a particles described herein can have a tap density of less than about: 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some cases, particles or compositions described herein can have a tap density of more than about 0.6 g/cm³, 0.7 g/cm³. In some cases, tap density can be a measure of the envelope mass density characterizing a particle. The envelope mass density of a particle of a statistically isotropic shape can be defined as the mass of the particle divided by the minimum sphere envelope volume within which it can be enclosed. Features which can contribute to low tap density include irregular surface texture, porous structure or a combination thereof. Tap density can be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.).

In some embodiments, particles of an active ingredient or a pharmaceutically acceptable salt thereof can be mixed in sizes. In some cases, the mixed sizes can change the release time of the drug. For example, encapsulated particles that comprise an additional enteric coating with small sizes (e.g. about 20 μm to about 40 μm) can be readily absorbed from the intestines into the blood stream while larger enteric coated, encapsulated particles larger than about 60 μm can take longer to be absorbed into the blood stream. In some cases, particles with diameters of about 20 μm to about 40 μm can absorb faster than particles with diameters of about 50 μm to about 200 μm. In some embodiments, the particles with sizes of about 50 μm to about 200 μm can be mixed with particles with sizes of about 20 μm to about 40 μm. In some embodiments, the weight to weight ratio of the particles with diameters of about 70 μm to about 100 μm to the particles with sizes of about 30 μm to about 60 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some embodiments, the weight to weight ratio of the particles with diameters of about 70 μm to about 100 μm to the particles with sizes of about 30 μm to about 60 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some embodiments, the particles with larger sizes (about 70 μm to about 200 μm) can be mixed with particles with smaller sizes (about 20 μm to about 40 μm). In some embodiments, the weight to weight ratio of the particles with larger sizes (about 70 μm to about 200 μm) to the particles with smaller sizes (about 20 μm to about 40 μm) can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10.

In some embodiments, active ingredient particles can be produced by spray drying. In some cases, encapsulated active ingredient particles can be produce by spray drying. In some cases, active ingredient particles with two or more coating layers (e.g., encapsulated with two or more layers) can be produce by spray drying. In some instances, active ingredient particles can be produced by another method. In some instances, active ingredient particles can be produced by air-jet micronization, spiral milling, controlled precipitation, high-pressure homogenization, or cryo-milling.

In some embodiments, the encapsulated active ingredient particles can be processed through a fluidized bed to apply an one or more additional coatings. For example, the encapsulated active ingredient particles can be processed through a fluidizer bed to apply an outer enteric coating.

In some embodiments, particle diameters can be measured by a particle analyzer using laser diffraction (LD), static light scattering, dynamic light scattering (DLS), or nanoparticle tracking analysis (NTA).

In some embodiments, particles that are not of a pharmaceutically acceptable excipient, can have particle diameters ranging from about 20 μm to about 70 μm. In some embodiments, particle diameters can be measured by a particle analyzer using laser diffraction (LD), static light scattering, dynamic light scattering (DLS), or nanoparticle tracking analysis (NTA).

### Packaging of the Powdery Compositions

In some embodiments, the composition can be contained within a food, a beverage, a liquid, a capsule, a tablet, a gel, a gummy, a spray, an ointment, a paste, a jelly, an oil, a butter, a tincture, a lotion, a cream, a balm, a syrup, or any combination thereof. In some embodiments, the composition is added to a food, a beverage, a liquid, a capsule, a tablet, a gel, a gummy, a spray, an ointment, a paste, a jelly, an oil, a butter, a tincture, a lotion, a cream, a balm, a syrup, or any combination thereof. The composition described herein, can be added as a food or beverage additive. For example, a microencapsulated, enteric coated cannabinoid such as CBD, THC, or THC Delta-8 can be added to a breakfast bar. In some cases, a food can comprise a candy, a baked item (e.g., a cake, a brownie, a bar, a cookie, etc.), a gummy, a chip, a potato chip, a fatty or oily food item, or any combination thereof. In some cases, a food can be a medical food. In some cases, a beverage can comprise, a carbonated beverage, a coffee, a tea, a soda, an alcoholic beverage or any combination thereof. In some cases, a food or beverage containing a cannabinoid can be administered as a pharmaceutical or as a supplement. In some cases, microencapsulation (e.g., with two or more coatings) of a cannabinoid can increase the solubility of a cannabinoid as a food or beverage additive. For example, a microencapsulated, enteric coated cannabinoid can increase the bioavailability to the human body as a food or drink. In some cases, microencapsulation with two or more coatings can mask the taste of a cannabinoid. In some cases, microencapsulation with two or more coatings can delay the release of a cannabinoid. For example, a cannabinoid can be released in the small intestine when it is encapsulated with at least one enteric coating. A microencapsulated, enteric coated cannabinoid can be added at any stage of preparing of a food or drink, for example a microencapsulated cannabinoid can be added prior to the baking of a cookie.

In some cases, a composition herein can comprise a liquid, an excipient, a carrier, a diluent, a food, a medical food, a capsule, a vitamin, an unsaturated fatty acid, an ester thereof, or a salt thereof, an organic salt, an inorganic salt, or any combination thereof.

In some embodiments, a microencapsulated particles comprising two or more shell layers as a food or beverage additive can have a mean, a median, or a mode particle diameter of less than about: 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles as a food or beverage additive have a mean, a median, or a mode particle diameter of more than about, or equal to about: 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles as a food or beverage additive have a mean, a median, or a mode particle diameter ranging from about: 20 μm to about 200 μm, 40 μm to about 70 μm, 30 μm to about 100 μm, 50 μm to about 150 μm, 60 μm to about 130 μm, 70 μm to about 160 μm, or 100 μm to about 200 μm.

In some embodiments, a composition herein can be contained within a capsule. In some embodiments, a composition herein can be in unit dose form. In some embodiments, a capsule may comprise a single-piece capsule, two-piece capsule, transparent capsule, non-transparent capsule, opaque capsule, slow-release capsule, extended-release capsule, standard-release capsule, rapid-release capsule, quick-release capsule, hard-shell capsule, soft gel capsule, gel capsule, hard gelatin capsule, soft gelatin capsule, animal-based capsule, vegetarian capsule, polysaccharide capsule, cellulose capsule, mucopolysaccharide capsule, tapioca capsule, hydroxypropylmethyl cellulose (HPMC) capsule, pullulan capsule, enteric capsule, uncoated capsule, coated capsule, capsule comprising titanium dioxide, fatty acids, waxes, shellac, plastics, pasticizers, glycerin, sorbitol, plant fibers, additives, preservatives, colorants, or any combination thereof.

In some embodiments, a capsule (e.g., a unit dose capsule) can have a different size. In some embodiments, the capsule size is: 000, 00, 0, 1, 2, 3, or 4. In some embodiments, the capsule size can be 000. In some embodiments, the capsule size can be 00. In some embodiments, the capsule size can be 0. In some embodiments, the capsule size can be 1. In some embodiments, the capsule size can be 2. In some embodiments, the capsule size can be 3. In some embodiments, the capsule size can be 4. In some embodiments, the capsule capacity varies from about 0.21 ml to about 1.37 ml.

In some embodiments, the powdery composition described herein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

In some embodiments, the composition can be contained within a unit dose. In some cases, the composition can be contained within a container or capsule, wherein the container or capsule can be loaded with about 10% to about 90% (by volume) with the powdery composition. In some cases, the capsule can be loaded with more than about, less than about, or equal to about: 15%, 20%, 25%, 30%, 31%, 32%, 33%, 34%, 35%, 36%, 37%, 38%, 39%, 40%, 45%, 50%, 55%, 60%, 65%, 70%, 75%, 80%, 85%, or 90% (by volume) with a composition described herein. In some embodiments, the container or capsule can be loaded with about 5% to about 20%, about 20% to about 25%, about 20% to about 30%, about 20% to about 40%, about 20% to about 50%, about 20% to about 60%, about 20% to about 65%, about 20% to about 70%, about 20% to about 75%, 25% to about 30%, about 25% to about 40%, about 25% to about 50%, about 25% to about 60%, about 25% to about 65%, about 25% to about 70%, about 25% to about 75%, about 25% to about 90%, about 30% to about 40%, about 30% to about 50%, about 30% to about 60%, about 30% to about 65%, about 30% to about 70%, about 30% to about 75%, about 30% to about 85%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, about 70% to about 75%, about 70% to about 90%, about 80% to about 90%, or 75% to about 100% (by volume) with a powdery composition.

In some embodiments, the content of container or capsule comprises less than about: 10%, 9%, 8%, 7%, 6%, 5%, 4%, 3%, 2% or 1% water by weight. In some embodiments, the content of the capsule comprises less than about 50%, about 40%, about 30%, about 25%, about 20%, about 10%, about 5%, or 1% water by weight.

In some embodiments, the total content of all gases in the container or capsule can be less than about: 10%, 9%, 8%, 7%, 6%, 5%, 4%, 3%, 2% or 1% water by weight. In some embodiments, the total content of all gases in the capsule can be less than about 50%, about 40%, about 30%, about 25%, about 20%, about 10%, about 5%, or about 1% water by weight.

In some embodiments, a container can be made of a plastic, a glass, a metal, or any suitable container material.

In some embodiments, the container or capsule further comprises, in the volume not occupied by the powdery composition, an inert gas. In some embodiments, the inert gas comprises an elemental gas, a compound gas, a noble gas, helium, neon, argon, krypton, xenon, oganesson, compounds of noble gas, purified argon, purified nitrogen, nitrogen or any combination thereof. In some embodiments, the inert gas comprises nitrogen. In some cases, the inert gas within a capsule can comprise at least about: 75%, 80%, 85%, 90%, or 95% of the gas on a volume to volume basis.

In some embodiments, the composition can be contained within a unit dose, container or capsule, wherein the delivery can be at least in part contained within a bottle, can, beverage container, or stick pack.

### Delivery of the Composition

In some embodiments, the administration of the pharmaceutical composition, a supplement, or the second therapeutic can be administered orally, intra nasally, intra ocular, anally, by injection, intra venously, intra muscularly, subcutaneously, intra peritoneally, trans dermally, or any combination thereof. In some embodiments, the powdery pharmaceutical composition, when orally administered, provides a time to peak plasma concentration (Tmax) of the active ingredient or the salt thereof. The time to peak plasma concentration (Tmax) of the active ingredient or the salt thereof can range from about 15 minutes to about 90 minutes. In some embodiments, the time to peak plasma concentration (Tmax) of the active ingredient or the salt thereof can range from about 15 minutes to about 45 minutes.

In some embodiments, the administration of the composition can be by a food or a beverage. In some embodiments, administration can be oral administration of a food or beverage.

In some embodiments, administering can be by oral ingestion, or topical application. In some embodiments, administering can comprise oral ingestion and the oral ingestion can comprise oral ingestion of a food, a beverage, a liquid, a gel, a capsule, or any combination thereof. In some embodiments, administering can comprise topical application and the topical application can comprise topical application of a lotion, a tincture, a balm, a cream, an oil, a gel, a butter, a liquid, a spray, an ointment, a paste, a jelly, or any combination thereof. In some embodiments, administering can be performed at least about: 1 time per day, 2 times per day, 3 times per day, 4 times per day, 5 times per day, 6 times per day or more than 6 times per day. In some cases, administering can be performed daily, weekly, monthly, or as needed. In some embodiments, administering can be conducted one, twice, three, or four times per day. In some cases, administration can be provided by a subject (e.g. the patient), a health care provider, or both.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years.

Also disclosed herein are kits comprising the composition contained at least in part in packaging (e.g., a container). It some cases, kits can be in unit dose form. Also disclosed herein are methods of making kits comprising a composition contained at least in part in packaging. In some cases, a kit can comprise a supplement disclosed herein. In some cases, a kit can comprise instructions for use of the composition.

Also disclosed herein are methods of treating or preventing a disease comprising treating or preventing the disease or condition by administering a therapeutically effective amount of the powdery pharmaceutical composition. Also disclosed herein are methods of treating or preventing a disease comprising treating or preventing the disease or condition by administering, (e.g., via oral administration) a therapeutically effective amount of a powdery pharmaceutical composition. In some embodiments, the disease can comprise treating or preventing a disease or condition selected from the group consisting of: a cancer, an anxiety, pruritus (itching), cognitive function, Alzheimer's disease, a chronic pain, pain management, multiple sclerosis, side effects of chemotherapy, AIDS, HIV, a neurodegenerative disorder, Tourette syndrome, cervical dystonia, a sleep disorder, an amyotrophic lateral sclerosis, an appetite disorder, a nausea associated with chemotherapy, a nausea, anorexia, spinal cord injury, glaucoma, an epilepsy, a seizure, an asthma, a substance dependency disorder (e.g. alcohol, cocaine, amphetamine, opioid), a psychiatric symptom, an autoimmune disease, an inflammation, and any combination thereof. In some cases, a disease or condition can comprise a posttraumatic stress disorder, cachexia, irritable bowel syndrome, a substance dependency disorder, a psychiatric symptom, an autoimmune disease, an inflammation, a sleep apnea, a headache, a migraine, an opioid addiction or any combination thereof. In some cases, a powdery pharmaceutical composition can be administered as a sleep aide, an appetite stimulant, for drug/alcohol dependency withdrawal or a combination thereof. In some embodiments, a cancer can be a breast cancer, a brain cancer, a tumor, a cervical cancer, a lung cancer, a prostate cancer, a pancreatic cancer, or any combination thereof. In some cases, a cancer can be a sarcoma, a melanoma, a lymphoma, a leukemia, or a combination thereof. In some cases, a disease can comprise neuropathic pain, pain, opioid addiction, opioid overdose, a heart disease, a hypertension, a sleep disorder, Guillain-Barre syndrome, Wilke's syndrome, a brain tumor, a human papillomavirus (HPV) infection, a brain injury (e.g. a traumatic brain injury), a depression, inflammation, Huntington's Disease, emesis, osteoporosis, schizophrenia, a cardiovascular disease, obesity, an infectious disease (bacterial, fungal, or viral), a metabolic syndrome-related disease, an arthritis, fibromyalgia, a dementia, Parkinson's disease or any combination thereof. In some cases, an arthritis can comprise osteoarthritis, fibromyalgia, rheumatoid arthritis, psoriatic arthritis, juvenile arthritis, gout, lupus. In some cases, a disease or condition can comprise pain, such as a chronic pain or an acute pain associated with an arthritis. In some cases, a disease or condition can comprise a pain associated with HIV, such as a chronic pain, an acute pain, or both. In some cases, a disease or condition can comprise inflammation associated with HIV. In some cases, a disease can comprise sickle cell disease. In some instances, sickle cell disease can comprise sickle cell anemia, sickle hemoglobin-C disease, sickle beta-plus thalassemia or sickle beta-zero thalassemia. In some cases, a disease or condition can comprise a pain (e.g., an acute pain or a chronic pain) associated with sickle cell anemia, sickle hemoglobin-C disease, sickle beta-plus thalassemia or sickle beta-zero thalassemia. In some cases, a disease or condition can comprise inflammation associated with sickle cell anemia, sickle hemoglobin-C disease, sickle beta-plus thalassemia or sickle beta-zero thalassemia. In some cases, a composition described herein such as a cannabinoid can be administered as an anti-inflammatory, an antimicrobial or both. In some cases, a composition described herein can alleviate symptoms associated with a disease. For example, a composition described herein can alleviate anemia, fatigue, pain, swelling (e.g., of hands and/or feet), infections, delayed growth, vision problems or any combination thereof. In some cases, CBD can bind to a fatty acid binding protein that transport anandamide intracellularly to Fatty Acid Amide Hydrolase (FAAH) for degradation, which may play a role in the inhibition of anandamide metabolism by CBD. In some instances, CBD can at least partially inhibit anandamide degradation. In some instances, CBD can reduce MAGL-mediated degradation of 2-AG. In some cases, a composition described herein such a cannabinoid (e.g. CBD or a salt thereof) can enhance the effect of another drug. For example, administration of CBD or a salt thereof can enhance the treatment of a cancer or increase the bioavailability of a drug. In some instances, CBD or a salt thereof can be a competitive inhibitor of cytochrome P450 and at least partially prevent cytochrome P450 from metabolizing other compounds. In some instances, a dose of an active ingredient may be decreased when administered with CBD. In some cases, a dose (by weight) of an active ingredient can be decreased by about: 5%, 10%, 20%, 30%, 40%, 50%, 60% 70%, 80% or 90% when administered with CBD.

In some cases, CBD, THC or both can comprise a cancer chemotherapeutic. For example, CBD, THC or both can be administered as a breast cancer chemotherapeutic. In some cases, a cannabinoid can be administered as a cancer chemotherapeutic. In some instances, CBD, THC or both can cause apoptosis of a cancer cell. In some instances, CBD, THC or both can elicit anti-neoplastic effect. In some cases, a cannabinoid such as CBD or THC may bind to a receptor on a cancerous cell. In some cases, a cannabinoid can at least partially bind to a G-protein coupled CB-receptor such as CB1-R and CB2-R. For example, THC may be a partial agonist for CG1-R, CB2-R or both. In another example, CBD may be an inverse agonist for CB1-R, CB2-R or both. In some instances, cannabinoids can at least partially inhibit cell cycle progress, at least partially inhibit cell growth, at least partially induce apoptosis, or any combination thereof of cancer cells. In some cases, a cannabinoid can at least partially inhibit migration of a cancer cell. In some instances, a cannabinoid can at least partially inhibit angiogenesis of cancer cells. In some cases, one or more cannabinoids can be administered as a cancer chemotherapeutic. In some cases, a cannabinoid can be administered in an amount of about: 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg as a cancer chemotherapeutic.

In some embodiments, prior to treating, a subject may have been diagnosed with the disease. In some embodiments, the subject may be a human, a man, a woman, an individual over 18 years of age, an individual under 18 years of age, or any combination thereof.

In some embodiments, a subject can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some embodiments, a method can further comprise diagnosing a subject as having the disease. In some embodiments, a diagnosing can comprise employing an in vitro diagnostic. In some embodiments, the in vitro diagnostic can be a companion diagnostic.

In some embodiments, a diagnosis can comprise a physical examination, a radiological image, a blood test, an antibody test, or any combination thereof. In some embodiments, a diagnosis can comprise a radiological image and the radiological image can comprise: a computed tomography (CT) image, an X-Ray image, a magnetic resonance image (MRI), an ultrasound image, or any combination thereof.

In some embodiments, a method can further comprise administering a second therapy to the subject. In some embodiments, a second therapy can comprise acetaminophen, a corticosteroid, an opioid, a nonsteroidal anti-inflammatory drug (NSAID), a COX-2 selective NSAID, a COX-2 inhibitor, methotrexate, hydroxychloroquine, prednisone, cortisone, a biological response modifier, a salt thereof, or any combination thereof. In some embodiments, a second therapy can comprise a biological response modifier and the biological response modifier can comprise: abatacept, adalimumab, adalimumab-atto, anakinra, certolizumab pegol, etanercept, etanercept-szzs, golimumab, infliximab, infliximab-dyyb, rituximab, sarilumab, tocilizumab, a biologically active fragment of any of these, a salt of any of these, or any combination thereof. In some embodiments, the second therapy can comprise a nonsteroidal anti-inflammatory drug and the nonsteroidal anti-inflammatory drug can comprise naproxen, ibuprofen, a salt of any of these, or any combination thereof. In some instances, a NSAID can comprise aspirin, diflunisal, dexibuprofen, oxaprozin, fenoprofen, indomethacin, tolmetin, celecoxib, clonixin, ketoprofen, salts thereof, or any combination thereof. In some instances, a COX-2 inhibitor can comprise etoricoxib, celecoxib, rofecoxib, valdecoxib, a salt thereof, or any combination thereof. In some cases, an active ingredient (e.g. THC oil) can be combined with another cannabinoid oil. In some embodiments, a composition can comprise an excipient, a diluent, a carrier, or any combination thereof.

In some embodiments, a bronchodilator can be administered before, concurrently or after administration of the powdery composition described herein (e.g. an encapsulated cannabinoid). In some cases, a bronchodilator can comprise a long acting or a short acting bronchodilator. In some instances, a bronchodilator can comprise a beta-2 antagonist, an anticholinergic, a xanthine derivative or a combination thereof. In some cases, a short acting bronchodilator can comprise albuterol, levalbuterol, pirbuterol, or a combination thereof. In some cases, a long acting bronchodilator can comprise salmeterol, formoterol, aclidinium, tiotropium, umeclidinium, or a combination thereof.

In some embodiments, a cannabinoid such as CBD can be administered with deoxycholic acid or a salt thereof. In some instances, deoxycholic acid or a salt thereof can increase bioavailability of a cannabinoid or a salt thereof. In some instances, a cannabinoid or a salt thereof can be administered concurrently or consecutively with deoxycholic acid or a salt thereof. In some instances, a cannabinoid or a salt thereof can be formulated into a composition with deoxycholic acid or a salt thereof.

In some embodiments, the composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some embodiments, the composition can be administered so that the active ingredient or the pharmaceutically acceptable salt thereof in the unit dose ranges from about: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg (milligram), 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range can be more than about, or equal to about: 10 μg, 25 μg, 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range can be less than about: 10 μg, 25 μg, 50 μg, 75 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. For example, tetrahydrocannabinol (THC), THC Isolate, Full Spectrum THC, THC Delta-7, THC Delta-8, THC Delta-9, THC Delta-10, THC Delta-11, Tetrahydrocannabivarin (THCV), Delta-13, and THC-THCA, and other cannabinoids can be administered in a unit dose form of about 0.25 mg, about 0.5 mg, about 1.0 mg, about 2.0 mg, about 2.5 mg, about 3.0 mg, about 4.0 mg, about 5.0 mg, about 6.0 mg, about 7.0 mg, about 8.0 mg, about 9.0 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 35 mg or 40 mg. Examples of compositions and methods of administration are shown in Table 3.

TABLE 3
Examples of compositions and methods of administration

| Active Pharmaceutical | Recommended | Route of |
|---|---|---|
| Ingredient | Dosing Level (THC) | Administration |
| Tetrahydrocannabinol | 2.5 mg | Oral administration |
| (THC), THC Isolate, Full | 5.0 mg | |
| Spectrum THC, THC | 10.0 mg | |
| Delta-8, THC Delta-9, | 20.0 mg | |
| THC Delta-11, | | |
| Tetrahydrocannabivarin | | |
| (THCV), Delta-13, and | | |
| THC-THCA, other | | |
| cannabinoids, or any | | |
| combination thereof | | |
| Delta-8 (Hemp derived) | 20 mg Delta-8 THC | Oral administration |
| and CBD (Full Spectrum) | and 10 mg CBD | |
| Delta-8 (Hemp derived) | 5 mg Delta-8 THC | Oral administration |
| and CBD (Full Spectrum) | and 5 mg CBD | |
| THC (Delta-9) and CBD | 10 mg Delta-9 THC | Oral administration |
| (Full Spectrum) | and 5 mg CBD | |
| | 20 mg Delta-9 THC | |
| | and 10 mg CBD | |
| THC (Delta-9) and CBD | 5 mg Delta-9 THC | Oral administration |
| (Isolate) | and 5 mg CBD | |
| | 20 mg Delta-9 THC | |
| | and 10 mg CBD | |

### Specific Embodiments

A number of compositions, and methods are disclosed herein. Specific exemplary embodiments of these compositions and methods are disclosed below

Embodiment 1. A powdery pharmaceutical composition, for oral use, comprising:
i) particles of a pharmaceutically acceptable excipient; and
ii) particles comprising an active ingredient or a pharmaceutically acceptable salt thereof at least partially encapsulated in a coating material and wherein the particles at least partially encapsulated in the coating material are spray dried, and wherein the encapsulated particles are at least partially encapsulated by an enteric coating in a fluid bed,
   wherein
   at least a portion of the particles of the pharmaceutically acceptable excipient have a particle diameter ranging from about 50 micrometers to about 200 micrometers, as measured by a particle size analyzer using laser diffraction;
   at least a portion of the particles comprising the active ingredient, or the pharmaceutically acceptable salt thereof encapsulated in the coating material have a particle diameter ranging from about 20 micrometers to about 200 micrometers, or about 40 micrometers to about 70 micrometers, as measured by a particle size analyzer using laser diffraction.

Embodiment 2. The powdery pharmaceutical composition of embodiment 1, wherein in a human clinical trial, the powdery pharmaceutical composition, when taken orally, provides in at least part of the humans in the clinical trial a time to peak plasma concentration (Tmax) of the active ingredient or the salt thereof ranging from about 15 minutes to about 90 minutes, or from about 15 minutes to about forty-five (45) minutes.

Embodiment 3. The powdery pharmaceutical composition of embodiment 1 or 2, wherein the powdery pharmaceutical composition is in unit dose form.

Embodiment 5. A powdery composition, for oral administration comprising:
i) particles of a pharmaceutically acceptable excipient; and
ii) a plurality of spray dried particles, each particle of the plurality of spray dried particles comprising a cannabinoid or a pharmaceutically acceptable salt thereof, substantially encapsulated in a coating material, and wherein the encapsulated particles are at least partially encapsulated by an enteric coating in a fluid bed, wherein
   within the plurality of spray dried particles comprising the cannabinoid, or the pharmaceutically acceptable salt thereof, substantially encapsulated in the coating material, and at least partially encapsulated by the enteric coating individually have a particle diameter ranging from about 40 micrometer to about 100 micrometers, as measured by a particle analyzer using laser diffraction and,
   wherein
a) The encapsulation coating material comprises a hydroxypropyl methylcellulose (HPMC), a hydroxypropyl methylcellulose acetate succinate (HPMCAS), a cyclodextrin, a maltodextrin, a povidone, a copovidone or any combination thereof, the enteric coating material comprises an additional polymer,
b) the encapsulation coating material does not comprise 1) diketopiperazine, 2) leucine, 3) trehalose, 4) distearoylphosphatidylcholine (DSPC) or a combination thereof and wherein
c) when the coating material comprises 1) it does not comprise 2)-4), when the coating material comprises 2) it does not comprise 1), 3), or 4), when the coating material comprises 3), it does not comprise 1), 2), or 4), and when the coating material comprises 4) it does not comprise 1)-3).

### EXAMPLES

The following examples are included for illustrative purposes only and are not intended to limit the scope of the disclosure.

### Example 1

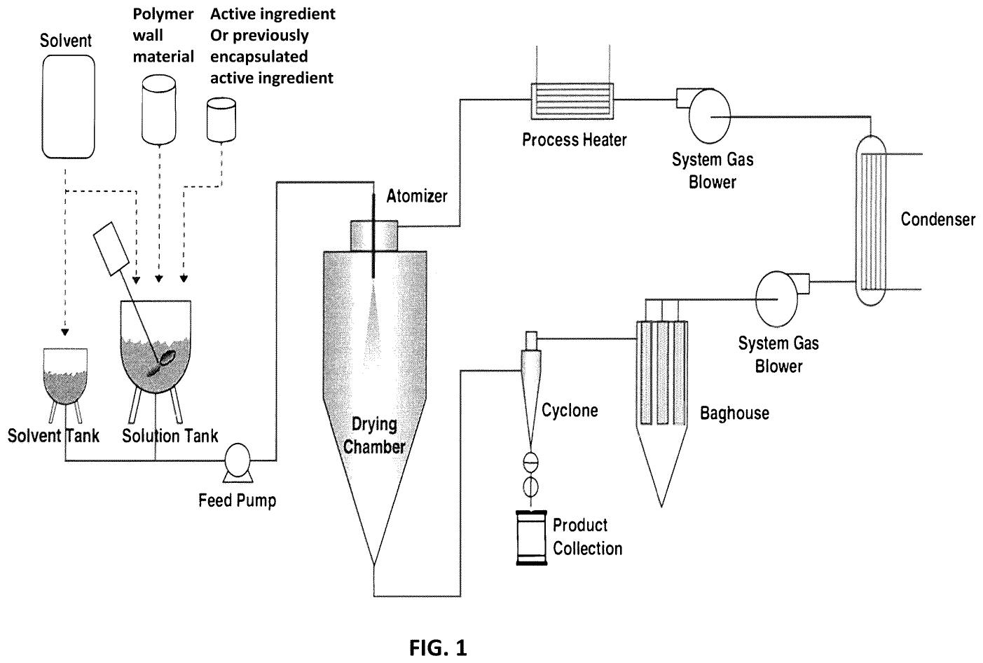

The active encapsulated ingredient (e.g. CBD) in a dry powdery composition described herein is manufactured by a spray drying system. FIG. 1 shows a spray drying manufacturing system for microencapsulated oils comprising a closed spray drying chamber which receives a solution comprising a polymer wall material in a suitable solvent mixed with oil droplets comprising the active ingredient (e.g. CBD) or previously encapsulated particles. Prior to spray drying, a polymer wall material (e.g. hydroxypropyl methylcellulose acetate succinate (HPMCAS) is dissolved in a solvent. The active ingredient oil (e.g. CBD) is made into small oil droplets with a micronizer prior to adding to the dissolved wall material and solvent. In some cases, a previously encapsulated cannabinoid is added to the dissolved wall material and solvent. The active ingredient (e.g. CBD oil droplets or the previously encapsulated active ingredient particles) are mixed thoroughly with the solvent and wall material to create a homogenous suspension of the active ingredient. The microencapsulated liquid suspension is fed into the atomizer. There is a spray nozzle at the top of the chamber, where the suspension is atomized with an inert gas. When organic solvents are used due to poor water solubility, Nitrogen drying gas can be used to prevent oxidation. The atomizer can be a two component (air/nitrogen and liquid), rotary, hydraulic (“pressure-type”), or ultrasonic nozzle types that distributes the suspension into fine droplets controlled by the atomizer pressure to achieve proper particle size for optimum absorption in the lung alveolars. The liquid feed is converted into small droplets by the atomizer and sprayed into a hot gas path that flash dries the droplets into solid particles. The solvent is evaporated and the particles are collected at the exit chamber. The drying chamber produces uniform fine particles that maintain tight particle size distribution. The particles are separated from the drying gas using a cyclone separator or filter bag to capture the resulting microencapsulated powder. The spray drying technology controls the particle size and particle size distribution. The process produces a consistent active ingredient particle size of about 30 micron to about 105 micron range.

The active encapsulated powder is fed into a fluid bed coating system in which a polymer enteric coating is applied to the powder. The enteric coating is separated from the drying process and captured in a cyclone separator or filter bag. The process produces consistent active ingredient particle size in the 40 micron to 70 micron range.

The active enteric coated powder is blended with an excipient carrier (lactose) product in a Patterson Kelly (PK Blender) and the blended powder as fed to a hopper. From the hopper, the dry powder was placed into a container. Blending of the CBD employs a V-type blender that has an intensifier bar that operates at high speeds to uniformly distribute the CBD and the carrier. The V-Blenders are manufactured by Patterson Kelly/PK Blender, Gemco or Ross blenders.

### Example 2

A male subject is diagnosed with chronic pain. The subject is prescribed a dosing regimen of a pharmaceutical composition. The pharmaceutical composition comprises a 2-layer encapsulated THC particle which is processed to a dry powder using the methods described herein (e.g., spay drying and fluid bed). The encapsulated THC is added to a beverage and is administered orally at a therapeutically effective dose.

### Example 3

A subject is diagnosed with anxiety. The subject is prescribed a dosing regimen of a pharmaceutical composition. The pharmaceutical composition comprises encapsulated THC with a three-layer coating and encapsulated CBD with a two-layer coating which is processed separately to dry powders using the methods described herein (e.g., spay drying and fluid bed). The THC and CBD is encapsulated with HPMCAS and with one (THC) layer or two layers (CBD) of an enteric coating. The THC is configured to release in the small intestine while the CBD is configured to release in the large intestine. The pharmaceutical composition is administered to the subject by a beverage. The dosing regimen comprises an effective amount of the combined THC and CBD encapsulated particles at a 1:1 ratio to treat the disease.

### Example 4

CBD (3.9 g) is dissolved in approximately 290 g of 200 proof ethanol. A separate solution of 1,2-distearoyl-sn-glycero-3-phosphocholine (1.5 g) in approximately 50 g of 200 proof ethanol is prepared, then added to the CBD solution. FDKP (9.6 g), approximately 50 g of ethanol, and 98 g of water is added to the CBD mixture. The resulting suspension is spray dried equipped with an inert loop, a dehumidifier, and a chiller. The spray drier is operated at 10% feed rate, 90% aspirator rate, 60 mm nitrogen flow rate, and an inlet temperature of 150° C. An additional coating of HPMCAS is added to the encapsulated CBD oil by a fluid bed. The resulting powder is analyzed by high performance liquid chromatography to determine percent CBD content.

### Example 5

Cannabidiol (CBD) was spray dried and microencapsulated in a first layer of HPMC (or HPMCAS) as disclosed herein. A second shell material (e.g., a second microencapsulation layer) was added to the microencapsulated CBD using methods disclosed herein. The second shell material comprised a polyvinylpyrrolidone (PVP), a povidone, a PVA (polyvinyl alcohol), Eudragit L30D, a liposome, a fatty acid, or a long chain triglyceride. The particles were larger than or equal to 20 micrometers.

### Example 6

Tetrahydrocannabinol (THC) was spray dried and microencapsulated in a first layer of HPMC (or HPMCAS) as disclosed herein. A second shell material (e.g., a second microencapsulation layer) was added to the microencapsulated THC using methods disclosed herein. The second shell material comprised polyvinylpyrrolidone (PVP), a povidone, a PVA (polyvinyl alcohol), Eudragit L30D, a liposome, a fatty acid, or a long chain triglyceride. The microencapsulated particles were larger than or equal to 20 micrometers.

### Example 7

Full spectrum CBD was spray dried and microencapsulated in a first layer of HPMCAS as disclosed herein. A second shell material (e.g., a second microencapsulation layer) comprising polyvinylpyrrolidone (PVP) was added to the microencapsulated CBD using methods disclosed herein. The microencapsulated particles were larger than or equal to 20 micrometers. The microencapsulated particles of CBD were added to water and mixed into a solution.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the methods presented in the disclosure. It is intended that the following claims define the scope of the disclosure and that methods and structures within the scope of these claims and their equivalents be covered thereby.

1. A powdery composition, comprising:
a) a plurality of encapsulated spray dried particles, wherein each particle of the plurality of spray dried particles is substantially encapsulated by a first coating, wherein the first coating is substantially encapsulated by one or more additional coatings; and
b) wherein the plurality of encapsulated spray dried particles comprise a cannabinoid or a pharmaceutically acceptable salt thereof, wherein
within the plurality of encapsulated spray dried particles, the plurality of encapsulated spray dried particles individually have a particle diameter ranging from about 20 micrometers to about 200 micrometers, or have a mean or median particle diameter of about 20 micrometers to about 200 micrometers, as measured by a particle analyzer using laser diffraction,
i) wherein the first coating comprises a hydroxypropyl methylcellulose (HPMC), a hydroxypropyl methylcellulose acetate succinate (HPMCAS), a cyclodextrin, a maltodextrin, a povidone, a copovidone or any combination thereof, and
ii) wherein the one or more additional coatings comprise an enteric coating.

2. The powdery composition of claim 1, wherein the enteric coating comprises a polymer.

3. The powdery composition of claim 1, wherein the enteric coating comprises a methyl methacrylate (MMA), a methyl acrylate-methacrylic acid copolymer, or both.

4. The powdery composition of claim 1, wherein the cannabinoid or the pharmaceutical acceptable salt thereof is present in an amount ranging from about 1 mg to about 100 mg.

5. The powdery composition of claim 1, wherein the cannabinoid or the pharmaceutical acceptable salt thereof is in the form of an oil.

6. The powdery composition of claim 1, wherein the cannabinoid or the pharmaceutically acceptable salt thereof comprises a tetrahydrocannabinol (THC) or a pharmaceutically acceptable salt thereof.

7. The powdery composition of claim 6, wherein the tetrahydrocannabinol (THC) or the pharmaceutical acceptable salt thereof comprises tetrahydrocannabinol Delta-8, tetrahydrocannabinol Delta-9, tetrahydrocannabinol Delta-10, tetrahydrocannabinol Delta-11, tetrahydrocannabinol Delta-13, tetrahydrocannabivarin (THCV), tetrahydrocannabinolic acid (THCA), full spectrum THC, broad spectrum THC, or a pharmaceutically acceptable salt thereof.

8. The powdery composition of claim 1, wherein the cannabinoid or the pharmaceutically acceptable salt thereof comprises a cannabidiol (CBD) or a pharmaceutically acceptable salt thereof.

9. The powdery composition of claim 1, wherein the cannabinoid or the pharmaceutically acceptable salt thereof comprises cannabigerol (CBG), cannabichromene (CBC), cannabidivarin (CBDV), cannabinol (CBN) a pharmaceutically acceptable salt of any of these, or any combination thereof.

10. The powdery composition of claim 1, further comprising a second cannabinoid or a pharmaceutically acceptable salt thereof.

11. The powdery composition of claim 1, wherein the powdery composition is in unit dose form.

12. The powdery composition of claim 1, comprising the first coating which comprises HPMCAS.

13. The powdery composition of claim 1, comprising the first coating which comprises HPMC.

14. The powdery composition of claim 1, comprising the first coating which comprises the cyclodextrin.

15. The powdery composition of claim 1, wherein the powdery composition further comprises an excipient, a carrier, a diluent, or any combination thereof.

16. A kit comprising the powdery composition of claim 1 and a container.

17. A liquid comprising the powdery composition of claim 1.

18. The liquid of claim 17, which is a beverage.

19. A dietary supplement comprising the powdery composition of claim 1.

20. A food comprising the powdery composition of claim 1.

21. The food of claim 20, which is a medical food.
